SEQUENCE LISTING (41 sequences)

SEQ ID NO: 1 - protein, 704 aa, Artificial Sequence
  Made in Lab - fusion protein construct
EVQLVESGGGLVQPGGSLRLSCAASGFTFSSYAMHWVRQAPGKGLEWVASISSTSGSKYYADSVKGRFTISRDNSKNTLYLQMNSLRAEDTAVYSCAKTYYDFWSGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASTKGPAGSGSGGSGSGAASTKGPQVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 449 aa, Artificial Sequence
  Made in Lab - fusion protein contruct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPAGSGSGGSGSGATVAAPSYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 704 aa, Artificial Sequence
  Made in Lab - fusion protein construct
QVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASTKGPAGSGSGGSGSGAASTKGPEVQLVESGGGLVQPGGSLRLSCAASGFTFSSYAMHWVRQAPGKGLEWVASISSTSGSKYYADSVKGRFTISRDNSKNTLYLQMNSLRAEDTAVYSCAKTYYDFWSGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 449 aa, Artificial Sequence
  Made in Lab - fusion protein contruct
SYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSTVAAPAGSGSGGSGSGATVAAPDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 699 aa, Artificial Sequence
  Made in Lab - fusion protein construct
EVQLVESGGGLVQPGGSLRLSCAASGFTFSSYAMHWVRQAPGKGLEWVASISSTSGSKYYADSVKGRFTISRDNSKNTLYLQMNSLRAEDTAVYSCAKTYYDFWSGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASTKGPAEAAAKAASTKGPQVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 444 aa, Artificial Sequence
  Made in Lab - fusion protein contruct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPAEAAAKATVAAPSYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 699 aa, Artificial Sequence
  Made in Lab - fusion protein construct
QVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASTKGPAEAAAKAASTKGPEVQLVESGGGLVQPGGSLRLSCAASGFTFSSYAMHWVRQAPGKGLEWVASISSTSGSKYYADSVKGRFTISRDNSKNTLYLQMNSLRAEDTAVYSCAKTYYDFWSGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 444 aa, Artificial Sequence
  Made in Lab - fusion protein contruct
SYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSTVAAPAEAAAKATVAAPDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 704 aa, Artificial Sequence
  Made in Lab - fusion protein construct
EVQLVESGGGLVQPGGSLRLSCAASGFTFSSYAMHWVRQAPGKGLEWVASISSTSGSKYYADSVKGRFTISRDNSKNTLYLQMNSLRAEDTAVYSCAKTYYDFWSGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASTKGPAEAAAKEAAAKAASTKGPQVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 449 aa, Artificial Sequence
  Made in Lab - fusion protein contruct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPAEAAAKEAAAKATVAAPSYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 704 aa, Artificial Sequence
  Made in Lab - fusion protein construct
QVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASTKGPAEAAAKEAAAKAASTKGPEVQLVESGGGLVQPGGSLRLSCAASGFTFSSYAMHWVRQAPGKGLEWVASISSTSGSKYYADSVKGRFTISRDNSKNTLYLQMNSLRAEDTAVYSCAKTYYDFWSGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 449 aa, Artificial Sequence
  Made in Lab - fusion protein contruct
SYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSTVAAPAEAAAKEAAAKATVAAPDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 709 aa, Artificial Sequence
  Made in Lab - fusion protein construct
EVQLVESGGGLVQPGGSLRLSCAASGFTFSSYAMHWVRQAPGKGLEWVASISSTSGSKYYADSVKGRFTISRDNSKNTLYLQMNSLRAEDTAVYSCAKTYYDFWSGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASTKGPAEAAAKEAAAKEAAAKAASTKGPQVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 454 aa, Artificial Sequence
  Made in Lab - fusion protein contruct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPAEAAAKEAAAKEAAAKATVAAPSYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 15 - protein, 709 aa, Artificial Sequence
  Made in Lab - fusion protein construct
QVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASTKGPAEAAAKEAAAKEAAAKAASTKGPEVQLVESGGGLVQPGGSLRLSCAASGFTFSSYAMHWVRQAPGKGLEWVASISSTSGSKYYADSVKGRFTISRDNSKNTLYLQMNSLRAEDTAVYSCAKTYYDFWSGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 454 aa, Artificial Sequence
  Made in Lab - fusion protein contruct
SYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSTVAAPAEAAAKEAAAKEAAAKATVAAPDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 462 aa, Artificial Sequence
  Made in Lab - fusion protein contruct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 462 aa, Artificial Sequence
  Made in Lab - fusion protein construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - protein, 478 aa, Artificial Sequence
  Made in Lab - fusion protein contruct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSEAAAKPEAAAKPEAAAKPDKTHTSPPSDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 20 - protein, 682 aa, Artificial Sequence
  Made in Lab - fusion protein construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 696 aa, Artificial Sequence
  Made in Lab - fusion protein contruct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSTEAAAKPEAAAKPEAAAKPDKTHTSPPSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 455 aa, Artificial Sequence
  Made in Lab - fusion protein contruct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 469 aa, Artificial Sequence
  Made in Lab - fusion protein construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSTEAAAKPEAAAKPEAAAKPDKTHTSPPSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 24 - protein, 689 aa, Artificial Sequence
  Made in Lab - fusion protein contruct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 689 aa, Artificial Sequence
  Made in Lab - fusion protein construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 705 aa, Artificial Sequence
  Made in Lab - fusion protein contruct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSEAAAKPEAAAKPEAAAKPDKTHTSPPSDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 446 aa, Artificial Sequence
  Made in Lab - fusion protein construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 28 - protein, 676 aa, Artificial Sequence
  Made in Lab - fusion protein construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 442 aa, Artificial Sequence
  Made in Lab - fusion protein contruct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 671 aa, Artificial Sequence
  Made in Lab - fusion protein construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 447 aa, Artificial Sequence
  Made in Lab - fusion protein contruct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPADIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 673 aa, Artificial Sequence
  Made in Lab - fusion protein construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSTEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 445 aa, Artificial Sequence
  Made in Lab - fusion protein construct
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 668 aa, Artificial Sequence
  Made in Lab - fusion protein construct
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSTVAAPQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 450 aa, Artificial Sequence
  Made in Lab - fusion protien construct
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 670 aa, Artificial Sequence
  Made in Lab - fusion protien construct
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSTVAAPSTQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 213 aa, Artificial Sequence
  Made in Lab - fusion protein construct
SYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 237 aa, Artificial Sequence
  Made in Lab - fusion protein construct
EVQLVESGGGLVQPGGSLRLSCAASGFTFSSYAMHWVRQAPGKGLEWVASISSTSGSKYYADSVKGRFTISRDNSKNTLYLQMNSLRAEDTAVYSCAKTYYDFWSGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSGSGHHHHHH

SEQ ID NO: 39 - protein, 673 aa, Artificial Sequence
  Made in Lab - fusion protein construct
QVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSGSGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 22 aa, Homo sapiens
MDMRVPAQLLGLLLLWLRGARC

SEQ ID NO: 41 - protein, 6 aa, Artificial Sequence
  Made in Lab - mutated IgG1 Fc fragment
LALAPG

(19) United States
(12) Patent Application Publication
(10) Pub. No.: US 2022/0064337 A1
(43) Pub. Date: Mar. 3, 2022

(54) ANTIGEN BINDING FORMATS FOR RECEPTOR COMPLEXES

(71) Applicant: Surrozen, Inc., South San Francisco, CA (US)

(72) Inventors: Yang LI, Mountain View, CA (US); Parthasarathy SAMPATHKUMAR, Foster City, CA (US)

(21) Appl. No.: 17/414,265

(22) PCT Filed: Dec. 19, 2019
(86) PCT No.: PCT/US2019/067661

§ 371 Date: Jun. 15, 2021

Related U.S. Application Data
(60) Provisional application No. 62/782,283, filed on Dec. 19, 2018.

(51) Int. Cl. C07K 16/46

(52) U.S. Cl. CPC C07K 16/468; C07K 2317/565; C07K 2317/53; C07K 2317/55

(57) ABSTRACT

The present invention provides multispecific multivalent antigen binding molecules that can function as surrogate molecules by binding and activating at least two receptors. The present invention provides a molecule comprising a plurality of antigen binding domains, wherein the binding domains bind to at least one first receptor and at least one second receptor, and related uses thereof.

### CROSS REFERENCE TO RELATED APPLICATIONS

This application claims priority to U.S. Provisional Application No. 62/782,283, filed Dec. 19, 2018, which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is SRZN_012_01WO_ST25.txt. The text file is about 187 KB, created on Dec. 18, 2019, and is being submitted electronically via EFS-Web.

### FIELD OF THE INVENTION

The present invention provides epitope binding formats having multi-specificity and/or multi-valency.

### BACKGROUND OF THE INVENTION

Antibodies are a well-established and rapidly growing drug class with at least 45 antibody-based products currently marketed for imaging or therapy in the United States and/or Europe with ˜$100 billion in total worldwide sales. This major clinical and commercial success with antibody therapeutics has fueled much interest in developing the next generation antibody drugs including bispecific antibodies. As their name implies, bispecific antibodies or multispecific antibodies (collectively “MsAb) bind to at least two different antigens, or at least two different epitopes on the same antigen, as first demonstrated more than 50 years ago. Engineering monospecific antibodies for multispecificity opens up many new potential therapeutic applications as evidenced by >30 BsAb in clinical development.

Bispecific or multispecific antibodies are a class of engineered antibody and antibody-like proteins that, in contrast to ‘regular’ monospecific antibodies, combine two or more different specific antigen binding elements in a single construct. Since bispecific antibodies do not typically occur in nature, they are constructed either chemically or biologically, using techniques such as cell fusion or recombinant DNA technologies. The ability to bind two or more different epitopes with a single molecule offers a number of potential advantages. One approach is to use the specificity of one arm as a targeting site for individual molecules, cellular markers or organisms, such as bacteria and viruses, while the other arm functions as an effector site for the recruitment of effector cells or delivery of molecular payloads to the target, such as drugs, cytokines or toxins. Alternatively, bispecific antibodies can be used to dual target, allowing detection or binding of a target cell type with much higher specificity than monospecific antibodies.

The modular architecture of immunoglobulins has been exploited to create a growing number (>60) of alternative MsAb formats (see, e.g., Spiess et al (2015) Mol. Immunol. 67:95-106). MsAb are classified here into five distinct structural groups: (i) bispecific IgG (BsIgG); (ii) IgG appended with an additional antigen-binding moiety; (iii) MsAb fragments; (iv) Multispecific fusion proteins; and (v) MsAb conjugates. Each of these different MsAb formats brings different properties in binding valency for each antigen, geometry of antigen-binding sites, pharmacokinetic half-life, and in some cases effector functions.

For antagonistic MsAbs antibodies, which represent the vast majority of the MsAb molecules in development, the geometry of the antigen binding modules is less critical. However, for agonistic MsAbs, these molecules need to faithfully mimic the activity of the natural ligand, the binding geometry could be crucial (see, e.g., Shi, et al. (2018) J. Biol. Chem. 293:5909-5919). One such signaling molecule is Wnt (“Wingless-related integration site”, “Wingless and Int-1”, or “Wingless-Int”).

Wnt ligands and their signals play key roles in the control of development, homeostasis, and regeneration of many essential organs and tissues, including bone, liver, skin, stomach, intestine, kidney, central nervous system, mammary gland, taste bud, ovary, cochlea, and many other tissues (reviewed, e.g., by Clevers, Loh, and Nusse (2014) Science; 346:54). Modulation of Wnt signaling pathways has potential for treatment of degenerative diseases and tissue injuries.

The seven-pass transmembrane receptor Frizzled (Fzd) is critical for nearly all Wnt signaling, and the N-terminal Fzd cysteine rich domain (CRD) serves as the Wnt binding domain. In addition to Fzd, the Wnt/β-catenin pathway requires the Low-density lipoprotein receptor related proteins 5 and 6 (Lrp5/6) to serve as co-receptors. LRP5 and LRP6 are functionally redundant single-pass transmembrane receptors. Biochemical studies of LRP6 indicate that different Wnts may bind to different extracellular domains of the LRP5/6 protein. The LRP6 extracellular domain contains four repeating sequences of β-propeller and epidermal growth factor-like (βP-E) domains. The crystal structures of the extracellular LRP6 regions indicate that the βP-E repeats represent two discrete, compact, rigid structures, each containing two βP-E pairs. Wnt9b binds the first two βP-E repeats on the extracellular domain of LRP6, whereas Wnt3a binds the last two βP-E domains. Non-Wnt agonists include Norrin and R-Spondin. Norrin is a Fz4-specific ligand that, in complex with LRP5 activates the Wnt signaling pathway. The four R-Spondin genes represent a family of conserved secreted proteins that potentiate the Wnt pathway. LGR4/5/6 (leucine-rich repeat-containing GPCRs 4, 5, and 6) are receptors for R-Spondins. The role of R-Spondins appears to stabilize the Wnt receptors, Fzd, and LRP6, to promote Wnt signaling.

Thus, a need exists to develop different antigen binding formats that mimic the binding of a natural ligand to co-receptor complexes that elicit agonistic biological activity, e.g. Fzd and LRP receptors. The present invention fulfills this need by providing flexible structures of MsAbs formats that bind to different receptors (co-receptors) and act as a mimetic of the natural ligand.

### SUMMARY OF THE INVENTION

The present invention provides a molecule comprising a plurality of antigen binding domains, wherein the binding domains bind to at least one first receptor and at least one second receptor, and related uses thereof. In certain embodiments, the molecule is either an agonist or antagonist. In embodiments that are agonists, the molecule mimics a natural ligand. In a further embodiment, the molecule binds to a receptor complex comprising at least one first receptor and one second receptor. In some embodiments, the first and second receptors are co-receptors. In some embodiments, at least one of the binding domains that binds to the first receptor is a Fab fragment. In further embodiments, binding domains that bind to the first and second receptor are both Fab fragments. In a certain embodiment, the Fab binding domains are attached in tandem to the N-terminus of an antibody Fc domain. In one embodiment, the molecule has a structure presented in FIG. 1A, 1B, 1C, or FIG. 6.

In certain embodiments, at least one first binding domain is fused directly to at least one second binding domain. In yet another embodiment the first binding domain is attached to the second binding domain with a peptide linker. In further embodiments, the peptide linker is about 1 amino acid in length to about 30 amino acids in length. In yet a further embodiment, the peptide linker is 5 amino acids in length to about 15 amino acids in length. In certain embodiments, the peptide linker comprises one or more glycine and/or serine residues.

In a related embodiment, the disclosure provides a molecule comprising at least two Fab binding domains and an Ig domain, wherein at least one Fab binding domain binds to at least one receptor of a Wnt co-receptor complex. In certain embodiments, the molecule comprises a first Fab binding domain that binds to a first receptor of the Wnt co-receptor complex and a second Fab binding domain that binds to a second receptor of the Wnt co-receptor complex. In some embodiments, the first Fab binding domain binds to at least one Fzd receptor and the second Fab binding domain binds to at least one LRP receptor. In some embodiments, the first Fab binding domain binds to at least one Fzd receptors selected from the group consisting of Fzd1, Fzd2, Fzd3, Fzd4, Fzd5, Fzd6, Fzd7, Fzd8, Fzd9, and Fzd 10. In some embodiments, the second Fab binding domain binds at least one LRP receptor selected from the group consisting from LRP5, LRP6, and LRP5/6. In some embodiments, the molecule is an agonist of Wnt signaling. In particular embodiments of the molecules disclosed herein, the molecule has a structure selected from the group consisting of the structures presented in FIG. 1A, 1B, 1C and FIG. 6.

In a related embodiments, the disclosure provides a first Fab binding domain that binds to a receptor of a Wnt co-receptor complex and a second Fab binding domain that binds to a non-Wnt receptor. In some embodiments, the molecule is an antagonist of Wnt signaling. In particular embodiments of the molecules disclosed herein, the molecule has a structure selected from the group consisting of the structures presented in FIG. 1A, 1B, 1C and FIG. 6.

In certain embodiments of the molecules disclosed herein, the first Fab binding domain (Inner Fab) is fused directly to the second Fab binding domain (Outer Fab). In certain embodiments, the Inner Fab is attached to the Outer Fab binding domain with a peptide linker. In certain embodiments, the peptide linker is selected from the group consisting of: a) a hinge-10 mer-hinge (HFL2); b) a hinge-helix-hinge (HHL); c) a 20 mer; d) an FcHinge; e) an upper hinge-helix-upper hinge; and f) a kappa hinge (khinge). In certain embodiments, the peptide linker comprises a linker sequence set forth in Table 1. In some embodiments, the peptide linker is about 1 amino acid in length to about 30 amino acids in length. In some embodiments, the peptide linker is about 5 amino acids in length to about 15 amino acids in length.

In a further related embodiments, the disclosure provides a polypeptide comprising or consisting of a polypeptide sequence having at least 70%, at least 80%, at least 90%, at least 95%, at least 98%, or at least 99% sequence identity to a sequence selected from SEQ ID NOs: 1-40. In some embodiments, the polypeptide comprises the CDR sequences set forth in any of SEQ ID NOs: 1-40, respectively.

In a related embodiments, the disclosure provides a molecule comprising two or more polypeptides comprising or consisting of a polypeptide sequence having at least 70%, at least 80%, at least 90%, at least 95%, at least 98%, or at least 99% sequence identity to a sequence selected from SEQ ID NOs: 1-40. In some embodiments, the polypeptides comprises the CDR sequences set forth in any of SEQ ID NOs: 1-40, respectively. In some embodiments, the molecule comprises four of such polypeptides. In some embodiments, two of the four polypeptides comprise an Ig or Fc region and the other two of the four polypeptides do not comprises an Ig or Fc region. In some embodiments, each of the four polypeptides comprise two Fab binding domains. In some of the embodiments, each of the four polypeptides comprises a first Fab binding domain that binds to a first receptor of the Wnt co-receptor complex and a second Fab binding domain that binds to a second receptor of the Wnt co-receptor complex. In some embodiments, the first Fab binding domain binds to at least one Fzd receptor and the second Fab binding domain binds to at least one LRP receptor.

In a related embodiment, the disclosure provides a polynucleotide encoding a polypeptide disclosed herein. In a related embodiment, the disclosure provides a cell comprising the polynucleotide. In certain embodiments, the two or more polypeptides are linked via covalent bonds, such as, e.g., disulfide bonds, and/or noncovalent interactions.

In another embodiment, the disclosure provides a method of agonizing the Wnt pathway, comprising contacting a cell with a molecule disclosed herein, wherein the molecule is a Wnt agonist.

In another embodiment, the disclosure provides a method of inhibiting the Wnt pathway, comprising contacting a cell with a molecule disclosed herein, wherein the molecule is a Wnt antagonist.

In another embodiment, the disclosure includes a composition, e.g., a pharmaceutical composition, comprising a molecule disclosed herein, optionally further comprising a pharmaceutically acceptable diluent, carrier, or excipient.

In another related embodiment, the disclosure provides a method of treating a disease or disorder where tissue regeneration is desired, comprising providing to a subject in need thereof a molecule disclosed herein, wherein the molecule is a Wnt agonist.

### BRIEF DESCRIPTION OF THE DRAWINGS

FIGS. 1A-1C: Schematic representation of (A) DFab-Ig; (B) DFabOC-Ig; and (C) DFabIC-Ig molecules. Linkers between the “outer Fab” and “inner Fab” domains are represented as dotted lines. Various domains are abbreviated as: VHo: VH domain of outer Fab; VLo: VL domain of outer Fab; VHi: VH domain of inner Fab; VLi: VL domain of inner Fab; CH1: IgG1 heavy-chain constant domain; CLo: CL domain of outer Fab; and CLi: CL domain of inner Fab.

FIG. 2: STF activity of eight different tetravalent bispecific DFab-Ig Wnt-agonist antibodies, containing the Fab domains of 1R07 and 10SA7, in the presence of 20 mM Rspo2 along with the “cells only” control in which no Wnt-agonists were added.

FIG. 3: STF activity of six different tetravalent bispecific DFabOC-Ig Wnt-agonist antibodies, containing the Fab domains of 18R5 and YW211.31.57, in the presence of 20 mM Rspo2 along with the “cells only” control in which no Wnt-agonists were added and an Wnt-agonist, R2M3-26.

FIG. 4: STF activity of six different tetravalent bispecific DFabIC-Ig Wnt-agonist antibodies, containing the Fab domains of 1R07 and 10SA7, in the presence of 20 mM Rspo2 along with the “cells only” control in which no Wnt-agonists were added and an Wnt-agonist, R2M3-26.

FIG. 5: Inhibitory effect of five different tetravalent bispecific DFabOC-Ig Wnt-antagonist antibodies, containing Fab domains of pembrolizumab (pembro) and 18R5 in different configurations, on the Wnt-activity induced by a Wnt-surrogate R2M3-26 in the presence of 20 mM Rspo2.

FIG. 6: Schematic representation of CrossFIT-Ig tetravalent bispecific antibody format. Linkers between the “outer Fab” and “inner Fab” domains are represented as purple-dotted lines. Various domains are abbreviated as: VHo: VH domain of outer Fab; VLo: VL domain of outer Fab; VHi: VH domain of inner Fab; VLi: VL domain of inner Fab; CH1: IgG1 heavy-chain constant domain; CLo: CL domain of outer Fab; and CLi: CL domain of inner Fab.

FIG. 7: STF activity of a tetravalent bispecific antibody designed in the CrossFIT-Ig format, containing the Fab domains of 1R07 and 10SA7, in the presence of 20 mM Rspo2 at concentration from 0.001 nm to 10 nM. Wnt signal activation was observed.

### DETAILED DESCRIPTION

As used herein, including the appended claims, the singular forms of words such as “a,” “an,” and “the,” include their corresponding plural references unless the context clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Each embodiment in this specification is to be applied mutatis mutandis to every other embodiment unless expressly stated otherwise.

All references cited herein are incorporated by reference to the same extent as if each individual publication, patent application, or patent, was specifically and individually indicated to be incorporated by reference.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, immunology, chemical syntheses, chemical analyses, pharmaceutical preparation, formulation, and delivery, and treatment of subjects. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

The terms “administering” or “introducing” or “providing”, as used herein, refer to delivery of a composition to a cell, to cells, tissues and/or organs of a subject, or to a subject. Such administering or introducing may take place in vivo, in vitro or ex vivo.

As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule. As used herein, the term encompasses not only intact polyclonal or monoclonal antibodies, but also fragments thereof (such as dAb, Fab, Fab′, F(ab′)2, Fv), single chain (scFv), V_{HH}, synthetic variants thereof, naturally occurring variants, fusion proteins comprising an antibody or an antigen-binding fragment thereof, humanized antibodies, chimeric antibodies, and any other modified configuration of the immunoglobulin molecule that comprises an antigen-binding site or fragment (epitope recognition site) of the required specificity. “Diabodies”, multivalent or multispecific fragments constructed by gene fusion (WO94/13804; P. Holliger et al (1993), Proc. Natl. Acad. Sci. USA 90 6444-6448) are also a particular form of antibody contemplated herein. Minibodies comprising a scFv joined to a CH3 domain are also included herein (See e.g., S. Hu et al. (1996), Cancer Res., 56:3055-3061; Ward, E. S. et al. (1989) Nature 341:544-546; Bird et al. (1988) Science 242:423-426; Huston et al. (1988) Proc. Natl. Acad. Sci. USA 85:5879-5883; PCT/US92/09965; WO94/13804; P. Holliger et al. (1993) Proc. Natl. Acad. Sci. USA 90:6444-6448; and Y. Reiter et al. (1996) Nature Biotech. 14:1239-1245).

The term “antigen-binding fragment” as used herein refers to a polypeptide fragment that contains at least one CDR of an immunoglobulin heavy and/or light chain, or of a V_{HH}, that binds to the antigen of interest, in particular to one or more Fzd receptor or LRP5 or LRP6 receptor. In this regard, an antigen-binding fragment of the herein described antibodies may comprise 1, 2, 3, 4, 5, or all 6 CDRs of a VH and VL sequence set forth herein from antibodies that bind one or more Fzd receptor or LRP5 and/or LRP6. In particular embodiments, an antigen-binding fragment may comprise all three VH CDRs or all three VL CDRs. Similarly, an antigen binding fragment thereof may comprise all three CDRs of a V_{HH} binding fragment. An antigen-binding fragment of a Fzd-specific antibody is capable of binding to a Fzd receptor. An antigen-binding fragment of a LRP5/6-specific antibody is capable of binding to a LRP5 and/or LRP6 receptor. As used herein, the term encompasses not only isolated fragments but also polypeptides comprising an antigen-binding fragment of an antibody disclosed herein, such as, for example, fusion proteins comprising an antigen-binding fragment of an antibody disclosed herein, such as, e.g., a fusion protein comprising a V_{HH} that binds one or more Fzd receptors and a V_{HH} that binds LRP5 and/or LRP6.

The term “antigen” refers to a molecule or a portion of a molecule capable of being bound by a selective binding agent, such as an antibody, and additionally capable of being used in an animal to produce antibodies capable of binding to an epitope of that antigen. In certain embodiments, a binding agent (e.g., a Wnt surrogate molecule or binding region thereof) is said to specifically bind an antigen when it preferentially recognizes its target antigen in a complex mixture of proteins and/or macromolecules. In certain embodiments, a Wnt surrogate molecule or binding region thereof (e.g., an antibody or antigen-binding fragment thereof) is said to specifically bind an antigen when the equilibrium dissociation constant is ≤10⁻⁷ or ≤10⁻⁸ M. In some embodiments, the equilibrium dissociation constant may be ≤10⁻⁹ M or ≤10⁻¹° M.

As used herein, the term “CDR” refers to at least one of the three hypervariable regions of a heavy or light chain variable (V) region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site. In certain embodiments, antibodies and antigen-binding fragments thereof as described herein include a heavy chain and a light chain CDRs, respectively interposed between a heavy chain and a light chain framework regions (FRs) which provide support to the CDRs and define the spatial relationship of the CDRs relative to each other.

As used herein, the term “FRs” refer to the four flanking amino acid sequences which frame the CDRs of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains. The structures and locations of immunoglobulin CDRs and variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), V_{HH}, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including Wnt surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”

The term “co-receptor” refers to a first cell surface receptor that binds signaling molecule or ligand in conjunction with another receptor to facilitate ligand recognition and initiate a biological process, such as Wnt pathway signaling.

The term “agonist activity” refers to the ability of an agonist to mimic the effect or activity of a naturally occurring protein binding to a first and second co-receptor.

As used herein “peptide linker” or “linker moiety” refers to a sequence of sometimes repeating amino acid residues, usually glycine and serine, that are used to join the various antigen binding domains described below. The length of the linker sequence determines the flexibility of the antigen binding domains in MsAbs, in particular, in the binding of epitopes on co-receptors such as Fzd receptors and LRP5 and/or LRP6.

As used herein, the term “enhances” refers to a measurable increase in the level of receptor signaling modulated by a ligand or ligand agonist compared with the level in the absence of the agonist, e.g., a Wnt surrogate molecule. In particular embodiments, the increase in the level of receptor signaling is at least 10%, at least 20%, at least 50%, at least two-fold, at least five-fold, at least 10-fold, at least 20-fold, at least 50-fold, or at least 100-fold as compared to the level of receptor signaling in the absence of the agonist, e.g., in the same cell type.

An antigen or epitope that “specifically binds” or “preferentially binds” (used interchangeably herein) to an antibody or antigen-binding fragment thereof is a term well understood in the art, and methods to determine such specific or preferential binding are also well known in the art. A molecule, e.g., a Wnt surrogate molecule, is said to exhibit “specific binding” or “preferential binding” if it reacts or associates more frequently, more rapidly, with greater duration and/or with greater affinity with a particular cell or substance than it does with alternative cells or substances. A molecule or binding region thereof, e.g., a Wnt surrogate molecule or binding region thereof, “specifically binds” or “preferentially binds” to a target antigen, e.g., a Fzd receptor, if it binds with greater affinity, avidity, more readily, and/or with greater duration than it binds to other substances. It is also understood by reading this definition that, for example, a surrogate molecule or binding region thereof that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al. (1986) Nucl. Acids Res. 14:9081; Stec et al. (1984) J. Am. Chem. Soc. 106:6077; Stein et al. (1988) Nucl. Acids Res. 16:3209; Zon et al. (1991) Anti-Cancer Drug Design, 6:539; Zon et al. (1991) Oligonucleotides and Analogues: A Practical Approach, pp. 87-108 (F. Eckstein, Ed.), Oxford University Press, Oxford England; Stec et al., U.S. Pat. No. 5,151,510; Uhlmann and Peyman (1990) Chem. Rev. 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

The term “host cell” is used to refer to a cell into which has been introduced, or which is capable of having introduced into it, a nucleic acid sequence encoding one or more of the herein described polypeptides, and which further expresses or is capable of expressing a selected gene of interest, such as a gene encoding any herein described polypeptide. The term includes the progeny of the parent cell, whether or not the progeny are identical in morphology or in genetic make-up to the original parent, so long as the selected gene is present. Accordingly there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. In one embodiment, the nucleic acid is integrated into the genome (e.g. chromosome) of the host cell. Integration may be promoted by inclusion of sequences which promote recombination with the genome, in accordance-with standard techniques.

“Transduction” also refers to the acquisition and transfer of eukaryotic cellular sequences by retroviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, MOLECULAR CLONING, A LABORATORY MANUAL, Cold Spring Harbor Laboratories; Davis et al., 1986, BASIC METHODS IN MOLECULAR BIOLOGY, Elsevier; and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell.

The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

The terms “polypeptide” “protein” and “peptide” and “glycoprotein” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass Wnt surrogate molecules, Fzd binding regions thereof, LRP5/6 binding regions thereof, antibodies and antigen-binding fragments thereof that bind to a Fzd receptor or a LRP5 or LRP6 receptor disclosed herein, or sequences that have deletions from, additions to, and/or substitutions of one or more amino acid of any of these polypeptides. Thus, a “polypeptide” or a “protein” can comprise one (termed “a monomer”) or a plurality (termed “a multimer”) of amino acid chains.

The term “isolated protein,” “or “isolated antibody” referred to herein means that a subject protein, surrogate molecule, or antibody: (1) is free of at least some other proteins with which it would typically be found in nature; (2) is essentially free of other proteins from the same source, e.g., from the same species, (3) is expressed by a cell from a different species; (4) has been separated from at least about 50 percent of polynucleotides, lipids, carbohydrates, or other materials with which it is associated in nature; (5) is not associated (by covalent or noncovalent interaction) with portions of a protein with which the “isolated protein” is associated in nature; (6) is operably associated (by covalent or noncovalent interaction) with a polypeptide with which it is not associated in nature; or (7) does not occur in nature. Such an isolated protein can be encoded by genomic DNA, cDNA, mRNA or other RNA, or may be of synthetic origin, or any combination thereof. In certain embodiments, an isolated protein may comprise naturally-occurring and/or artificial polypeptide sequences. In certain embodiments, the isolated protein is substantially free from proteins or polypeptides or other contaminants that are found in its natural environment that would interfere with its use (therapeutic, diagnostic, prophylactic, research or otherwise).

### II. General

The present invention provides structures of antigen binding molecules that act as surrogate molecules by binding to and modulating co-receptor signaling, for example, antigen binding molecules that bind to one or more Fzd receptor and one or more LRP5 or LRP6 receptor and modulate a downstream Wnt signaling pathway. In particular embodiments, the surrogate molecules activate or increase a signaling pathway associated with one or both of co-receptors. In particular embodiments, the surrogate molecules disclosed herein comprise: (i) one or more antibodies or antigen-binding fragments thereof that specifically bind to one or more first co-receptor, including antibodies or antigen-binding fragments thereof having particular co-receptor specificity and/or functional properties; and (ii) one or more antibodies or antigen-binding fragments thereof that specifically bind to one or more second co-receptors. Certain embodiments encompass specific structural formats or arrangements of the first and second co-receptor binding region(s) of the surrogate molecules advantageous in increasing downstream signaling and related biological effects.

Sequences of illustrative antibodies, or antigen-binding fragments, or complementarity determining regions (CDRs) thereof, that bind to one or more Fzd receptors, are set forth in the U.S. provisional applications Nos. 62/607,877 and 62/680,508, titled Anti-Frizzled Antibodies and Methods of Use, Attorney docket nos. SRZN-004/00US and SRZN-004/01US, filed on Dec. 19, 2017 and Jun. 4, 2018, respectively, and PCT Application Publication No. WO2019/126399. Sequences of illustrative LRP5 and/or LRP6 antibodies, or antigen-binding fragments, or complementarity determining regions (CDRs) thereof, are set forth in the U.S. provisional application Nos. 62/607,879 and 62/680,515, titled Anti-LRP5/6 Antibodies and Methods of Use, Attorney docket no. SRZN-005/00US and SRZN-005/01US, filed on Dec. 19, 2017 and Jun. 4, 2018, respectively, and PCT Application Publication No. WO2019/126401. Sequences of antigen binding molecules that bind one or more Fzd receptor and LRP5 and/or LRP6 are set forth in U.S. Provisional application Nos. 62/607,875, 62/641,217, and 62/680,522, titled Wnt Signaling Pathway Agonists, filed Dec. 19, 2017, Mar. 9, 2018, and Jun. 4, 2018, respectively, and PCT Application Publication No. WO2019/126398.

Antibodies and antibody fragments thereof may be prepared by methods well known in the art. For example, the proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present invention can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH: VL heterodimer including an antigen-binding site which retains much of the antigen recognition and binding capabilities of the native antibody molecule. (See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096).

Where bispecific antibodies are to be used, these may be conventional bispecific antibodies, which can be manufactured in a variety of ways (Holliger, P. and Winter G. (1993) Curr. Op. Biotechnol. 4:446-449), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knob s-into-holes engineering (J. B. B. Ridgeway et al. (1996) Protein Eng., 9:616-621).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, the antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al. (1988) Proc Natl Acad Sci USA 86:10029-10033; and Riechmann et al. (1988) Nature 332:323-327). Illustrative methods for humanization of the anti-Fzd antibodies disclosed herein include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856. Riechmann, L., et al., (1988) supra; Verhoeyen, M., et al., (1988) Science 239:1534-1536; Kettleborough, C. A., et al., (1991) Protein Engg 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technol. 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

### III. Structures of Receptor Surrogate Ligands

The disclosure provides, in certain aspects, multi-specific surrogate molecules that bind a first receptor (e.g., Fzd) and a second receptor (e.g., LRP 5/6), e.g., co-receptors. In particuarle embodiments, the coreceptors, or a domain or region thereof, are located on a cell surface, and in certain embodiments, the molecules bind an extracellular domain of the coreceptors. In certain embodiments, the molecule binds one or more Fzds and/or one or more LRPs. Surrogate molecules of the present invention are biologically active in binding to one or more of a first receptor and to one or more of a second receptor, and as an example, in the activation of Wnt signaling, the Wnt surrogate molecule is referred to as a Wnt agonist. The term “agonist” or “agonist activity” refers to the ability of an agonist molecule to mimic the effect or activity of a naturally occurring protein binding to a first and second receptor. The ability of the surrogate molecules and other receptor agonists disclosed herein to mimic the activity of the natural ligand could be confirmed by a number of assays. As an example, Wnt surrogate molecules, some of which are disclosed herein activate, enhance or increase the canonical Wnt/β-catenin signaling pathway.

In particular embodiments, the structures of the surrogate molecules disclosed herein are bispecific, i.e., they specifically bind to two or more different epitopes, e.g., one or more of a first receptor, and one or more of a second receptor. For example, a Wnt surrogate molecule can bind one or more human Fzd receptors and one or both of a human LRP5 and/or a human LRP6. In certain embodiments, a surrogate molecule is capable of modulating or modulates signaling events associated with at least one of the co-receptors in a cell contacted with the surrogate molecule. In certain embodiments, the surrogate molecule increases receptor signaling. As an example, a Wnt surrogate molecule specifically modulates the biological activity of a human Wnt/β-catenin signaling pathway.

In particular embodiments, surrogate molecules disclosed herein are multivalent, e.g., they comprise two or more regions that each specifically bind to the same epitope, e.g., two or more regions that bind to an epitope within one or more first co-receptor and/or two or more regions that bind to an epitope within a second co-receptor. In particular embodiments, they comprise two or more regions that bind to an epitope within a first co-receptor and two or more regions that bind to an epitope within a second co-receptor. In certain embodiments, surrogate molecules comprise a ratio of the number of regions that bind one or more first co-receptor to the number of regions that a second co-receptor of or about: 1:1, 2:1, 3:1, 4:1, 5:1, 6:1, 2:3, 2:5, 2:7, 7:2, 5:2, 3:2, 3:4, 3:5, 3:7, 3:8, 8:3, 7:3, 5:3, 4:3, 4:5, 4:7, 4:9, 9:4, 7:4, 5:4, 6:7, 7:6, 1:2, 1:3, 1:4, 1:5, or 1:6. In certain embodiments, the surrogate molecules are bispecific and multivalent.

The structures of the surrogate molecules disclosed herein may have any of a variety of different structural formats or configurations. The surrogate molecules may comprise polypeptides and/or non-polypeptide binding moieties, e.g., small molecules. In particular embodiments, the surrogate molecules comprise both a polypeptide region and a non-polypeptide binding moiety. In certain embodiments, the surrogate molecules may comprise a single polypeptide, or they may comprise two or more, three or more, or four or more polypeptides. In certain embodiments, one or more polypeptides of a surrogate molecule are antibodies or antigen-binding fragments thereof. In certain embodiments, surrogates comprise two antibodies or antigen binding fragments thereof, one that binds one or more first co-receptor and one that binds on or more second co-receptor. In certain embodiments, the surrogates comprises one, two, three, or four polypeptides, e.g., linked or bound to each other or fused to each other.

When the surrogate molecules comprise a single polypeptide, they may be a fusion protein comprising one or more first co-receptor binding domain and one or more second co-receptor binding domain. The binding domains may be directly fused or they may be connected via a linker, e.g., a polypeptide linker, including but not limited to any of those disclosed herein.

When the surrogate molecules comprise two or more polypeptides, the polypeptides may be linked via covalent bonds, such as, e.g., disulfide bonds, and/or noncovalent interactions. For example, heavy chains of human immunoglobulin IgG interact at the level of their CH3 domains directly, whereas, at the level of their CH2 domains, they interact via the carbohydrate attached to the asparagine (Asn) N84.4 in the DE turn. In particular embodiments, the surrogate molecules comprise one or more regions derived from an antibody or antigen-binding fragment thereof, e.g., antibody heavy chains or antibody light chains or fragments thereof. In certain embodiments, a surrogate polypeptide comprises two antibody heavy chain regions (e.g., hinge regions) bound together via one or more disulfide bond. In certain embodiments, a surrogate polypeptide comprises an antibody light chain region (e.g., a CL region) and an antibody heavy chain region (e.g., a CH1 region) bound together via one or more disulfide bond.

Surrogate polypeptides may be engineered to facilitate binding between two polypeptides. For example, Knobs-into-holes amino acid modifications may be introduced into two different polypeptides to facilitate their binding. Knobs-into-holes amino acid (AA) changes is a rational design strategy developed in antibody engineering, used for heterodimerization of the heavy chains, in the production of bispecific IgG antibodies. AA changes are engineered in order to create a knob on the CH3 of the heavy chains from a first antibody and a hole on the CH3 of the heavy chains of a second antibody. The knob may be represented by a tyrosine (Y) that belongs to the ‘very large’ IMGT volume class of AA, whereas the hole may be represented by a threonine (T) that belongs to the ‘small’ IMGT volume class. Other means of introducing modifications into polypeptides to facilitate their binding are known and available in the art. For example, specific amino acids may be introduced and used for cross-linking, such as Cysteine to form an intermolecular disulfide bond.

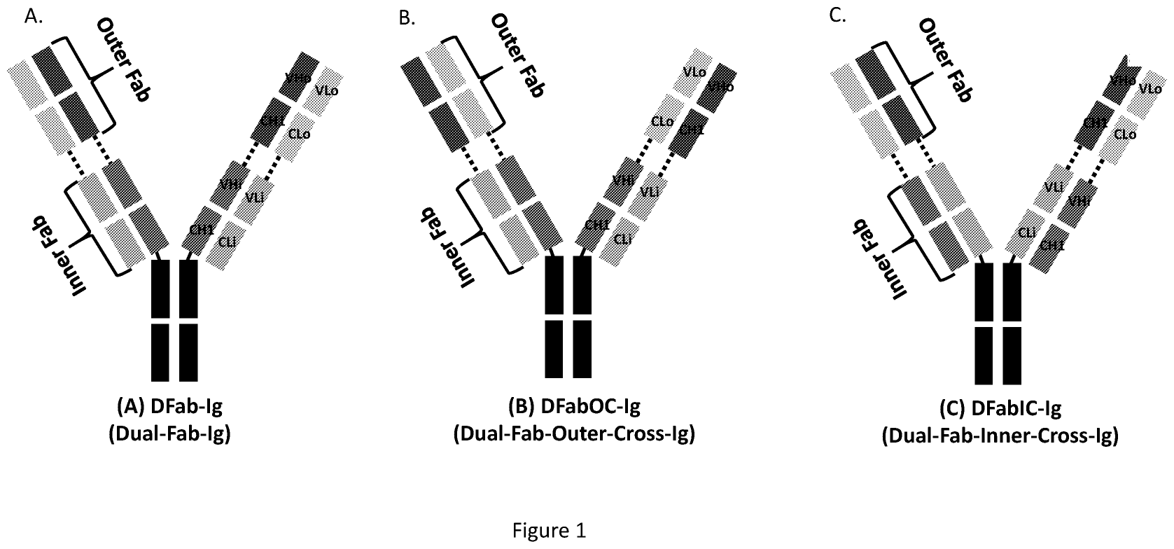

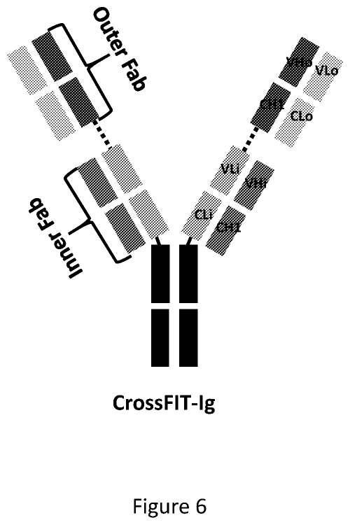

As depicted in FIGS. 1A-1C and 6, the molecule, comprises two Fab or antigen-binding fragments thereof that bind one or more first co-receptor and/or two Fab or antigen-binding fragments thereof that bind to one or more second co-receptor. In further embodiments, one or more of the Fab is present in a full IgG format, and in certain embodiments, both Fab are present in a full IgG format. In certain embodiments, the Fab in full IgG format specifically binds one or more first receptor (e.g., one or more Fzd receptor), and the other Fab specifically binds at least one second receptor (e.g., LRP5 and/or LRP6). For example, the Fab specifically binds one or more Fzd receptor, and the Fab in full IgG format specifically binds LRP5 and/or LRP6. In certain embodiments, the Fab specifically binds LRP5 and/or LRP6, and the Fab in full IgG format specifically binds one or more Fzd receptor. In certain embodiments, the Fab is fused to the N-terminus of the IgG, e.g., to the heavy chain or light chain N-terminus, optionally via a linker. In certain embodiments, the Fab is fused to the N-terminus of the heavy chain of the IgG and not fused to the light chain. In particular embodiments, the two chains of each Fab can be fused together directly or via a linker.

In various embodiments, including but not limited to those depicted in FIGS. 1A-1C and 6, a surrogate molecule comprises one or more Fab or antigen-binding fragment thereof. In certain embodiments, a surrogate molecule comprises two or more Fab or antigen-binding fragments thereof. In certain embodiments, a surrogate molecule comprises a first Fab fragment that specifically binds one or more Fzd receptor, and a second Fab that specifically binds LRP5 and/or LRP6. In certain embodiments, the first Fab specifically binds LRP5 and/or LRP6, and the second Fab specifically binds one or more Fzd receptor. This general structure, depicted in FIG. 1A, is referred to as Dual-Fab-Ig (DFab-Ig). In further embodiments, one Fab fragment is rotated such that the light chains of one Fab are directly fused or linked to the heavy chains of the second Fab. As illustrated in FIGS. 1B and 1C, when the second or “outer” Fab is rotated, the molecule is known as a Dual-Fab-Outer-Cross-IG (DFabOC-Ig), and when the Fab is rotated, the molecule is known as a Dual-Fab-Inner-Cross-Ig (DFabIC-Ig). The chains of the second Fab may be fused directly to the first Fab, or may be attached covalently with linkers between each Fab chain. In a further embodiment, the inner or first Fab is rotated such that only the inner or first Fab light chains are linked to the heavy chains of the outer or second Fab (CrossFIT-Ig; FIG. 6).

In certain embodiments, a molecule comprises four polypeptides, wherein each of the four polypeptides may be linked via covalent bonds, such as, e.g., disulfide bonds, and/or noncovalent interactions to at least one of the other three polypeptides. In some embodiments, the molecule comprises four polypeptides, each comprising two binding regions, e.g., Fab regions, wherein one of the binding regions binds one or more Fzd receptors, and the other binding region binds a Lrp5 or Lrp6 receptor. In certain embodiments, only two of the four polypeptides further comprises an full Ig or Fc domain, wherein the two polypeptides comprising the Ig or Fc domain bind each other, and wherein each of the other two polypeptides not comprising the full Ig or Fc domain each bind to a different one of the two polypeptides comprising the full Ig or Fc domain. In particular embodiments, the two binding domains of each polypeptide are fused to each other via a linker. In certain embodiments, the molecule comprises four polypeptides disclosed in any of SEQ ID NOs:1-40, or a variant thereof having at least 80%, at least 90%, at least 95%, at least 98%, or at least 99% sequence identity to a sequence disclosed in any of SEQ ID NOs:1-40. In particular embodiments, the variant retains all of the CDR sequences present in the corresponding sequence disclosed in any of SEQ ID NOs:1-40. In certain embodiments, the variant retains at least 70%, at least 80%, or at least 90% activity as the corresponding sequence, optionally in the context of the molecule.

Fabs may be converted into a full IgG format that includes both the Fab and Fc fragments, for example, using genetic engineering to generate a fusion polypeptide comprising the Fab fused to an Fc region, i.e., the Fab is present in a full IgG format. See FIGS. 1A-1C and 6. The Fc region for the full IgG format may be derived from any of a variety of different Fcs, including but not limited to, a wild-type or modified IgG1, IgG2, IgG3, IgG4 or other isotype, e.g., wild-type or modified human IgG1, human IgG2, human IgG3, human IgG4, human IgG4Pro (comprising a mutation in core hinge region that prevents the formation of IgG4 half molecules), human IgA, human IgE, human IgM, or the modified IgG1 referred to as IgG1 LALAPG. The L235A, P329G (LALA-PG) variant has been shown to eliminate complement binding and fixation as well as Fc-γ dependent antibody-dependent cell-mediated cytotoxicity (ADCC) in both murine IgG2a and human IgG1. These LALA-PG substitutions allow a more accurate translation of results generated with an “effectorless” antibody framework scaffold between mice and primates. In particular embodiments of any of the IgG disclosed herein, the IgG comprises one or more of the following amino acid substitutions: N297G, N297A, N297E, L234A, L235A, or P236G.

Non-limiting examples of bivalent and bispecific surrogate molecules of co-receptors that are bivalent towards both the one or more first receptor and one or more second receptor (e.g., Fzd and LRP) are provided as the structures depicted in FIGS. 1A-1C, and 6, where the first Fab is labeled as the “outer Fab” and the second Fab is labeled as the “inner Fab”. As shown, the Fabs may be fused in tandem to the N-terminus of the Ig (depicted in black), In particular embodiments, the Fab is described herein or comprises any of the CDR sets described herein (See Table 1).

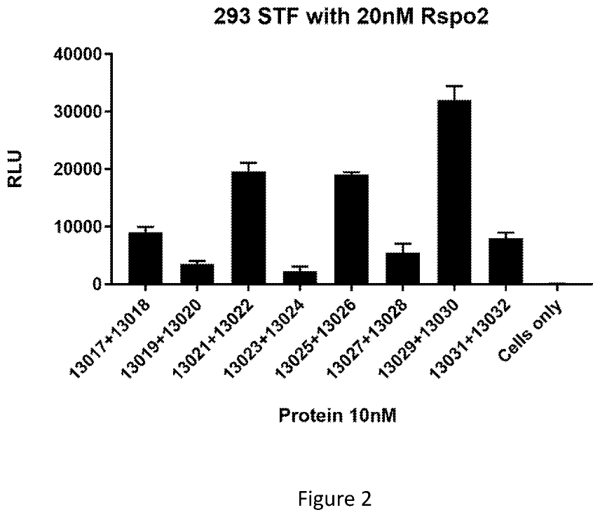

In certain embodiments, the antigen binding molecules have a format as described in PCT Application Publication No. WO2017/136820, e.g., a Fabs-in-tandem IgG (FIT-IG) format. FIT-IG also include the formats disclosed in, e.g., Gong, et al (2017) mAbs 9: 118-1128. In certain embodiments, FIT-Igs combine the functions of two antibodies into one molecule by re-arranging the DNA sequences of two parental monoclonal antibodies into two or three constructs and co-expressing them in mammalian cells. Examples of FIT-IG formats and constructs are provided in FIGS. 1A and 1B and FIGS. 2A and 2B of PCT Application Publication No. WO2017/136820. In certain embodiments, FIT-IGs require no Fc mutation; no scFv elements; and no linker or peptide connector. The Fab-domains in each arm work “in tandem” forming a tetravalent bi-specific antibody with four active and independent antigen binding sites that retain the biological function of their parental antibodies In particular embodiments, Wnt surrogates comprises a Fab and an IgG. In certain embodiments, the Fab binder LC is fused to the HC of the IgG, e.g., by a linker of various length in between. In various embodiment, the Fab binder HC can be fused or unfused to the LC of the IgG. A variation of this format has been called Fabs-in-tandem IgG (or FIT-Ig).

In certain embodiments the Wnt surrogate molecules have a format described in PCT Application Publication No. WO2009/080251 (Klein et al.), e.g. a CrossMab format. CrossMabs formats are also described in Schaefer et al. (2011) Proc. Natl. Acad. Sci USA 108:11187-11192. The CrossMab format allows correct assembly of two heavy chains and two light chains derived from existing antibodies to form a bispecific, bivalent IgG antibodies. The technology is based on the cross over the antibody domain within one Fab-arm of a bispecific IgG antibody in order to enable correct chain association. Various portions of the Fab can be exchanged, e.g., the entire Fab, the variable heavy and light chains, or the CH1-CL chains can be exchanged.

In further embodiments of the present invention, the FiT-Ig and CrossMab technologies are combined to create a multispecific, multivalent antigen binding molecule, Cross-FiT, as depicted in FIGS. 1A-1C and 6. Also contemplated is a linker between the crossed CL domain of the Fab and the Ig domains rather than between the CH1 and Ig domains. Additional antigen binding fragments, e.g., Fabs, VHH/sdAbs, and/or scFvs, can be appended to the Cross-FiT structure at various sites, e.g., the heavy or light chains and/or the C-terminus of the Fc domain to create multispecific antibodies.

In certain embodiments, the antigen binding formats are surrogate molecules that comprise one or more polypeptides comprising two or more binding regions. For illustrative purposes, the two or more binding regions may be a first receptor binding regions or a second receptor binding regions, or they may comprise one or more first receptor binding region and one or more second receptor binding region. The binding regions may be directly joined or contiguous, or may be separated by a linker, e.g. a polypeptide linker, or a non-peptidic linker, etc. The length of the linker, and therefore the spacing between the binding domains can be used to modulate the signal strength, and can be selected depending on the desired use of the surrogate and/or antagonist molecule. Specific examples of such linkers are provided in Table 1. Certain embodied linkers for the DFab-Ig structure, contain 2 antibody hinge regions separated by a given length of amino acid, yield a flexible linker which are designated “HFL” or “kHFL”. In other embodiments the 2 hinge regions are separate by various helical structures and designated “HHL” or “kHHL”.

The HFL2 linker consists of two linkers specific for the VH chains (see, e.g., Jakob, et al. (2013) mAbs 5(3):358-363) flanking a 10 mer of glycine and serine residues. Similarly, the kHFL2 consists of linker specific for the VL chains, specifically kappa chains (see, e.g., Jakob, et al. (2013) mAbs 5(3):358-363) also flanking a 10 mer of glycine and serine residues.

The HHL linkers consists of the VH chain specific linkers described above, flanking either 1, 2, 3 sets if repeating amino acids that form helical structures (see, e.g., Chen et al (2013) Adv Drug Deliv Rev. 65(10):1357-1369). Similarly, the kHHL linkers consist of the VL kappa chain specific linkers described above flanking 1, 2, or 3 sets of helical sequences (see, e.g., Chen et al (2013) supra).

For the DFabOC-Ig and DFabIC-Ig embodiments, linkers include a 20 mer peptide linker of 2 G4S repeats (see Chen et al. (2013) supra), a Fc hinge sequence corresponding to the lower hinge region of an Ig closest to the Fc domain of the Ig, the Upper Hinge corresponding to the hinge region sequence closest to the Fab domains, the Upper Hinge flanking 3 repeating helical structures, and the kappa linker and Upper Hinge flanking 3 repeating helical structures.

In embodiments, the enforced distance between binding domains can vary, but in certain embodiments may be less than about 100 angstroms, less than about 90 angstroms, less than about 80 angstroms, less than about 70 angstroms, less than about 60 angstroms, or less than about 50 angstroms. In some embodiments the linker is a rigid linker, in other embodiments the linker is a flexible linker. In certain embodiments where the linker is a peptide linker, it may be from about 1, 2, 3, 4, 5, 6, 7, 8, 9, 10, 11, 12, 13, 14, 15, 16, 17, 18, 19, 20 21, 22, 23, 24, 25, 26, 27, 28, 29, 30 or more amino acids in length, and is of sufficient length and amino acid composition to enforce the distance between binding domains. In some embodiments, the linker comprises or consists of one or more glycine and/or serine residues.

The surrogate molecule can be multimerized, e.g. through an Fc domain, by concatenation, coiled coils, polypeptide zippers, biotin/avidin or streptavidin multimerization, and the like. The surrogate molecules can also be joined to a moiety such as PEG, Fc, etc., as known in the art to enhance stability in vivo.

In certain embodiments, a surrogate molecule enhances or increases the co-receptors pathway signaling, e.g., in the case of Wnt-β-catenin signaling, by at least 30%, 35%, 40%, 45%, 50%, 60%, 70%, 75%, 80%, 85%, 90%, 95%, 100%, 110%, 150%, 200%, 250%, 300%, 400% or 500%, as compared to the β-catenin signaling induced by a neutral substance or negative control as measured in an assay described above, for example as measured in the TOPFIash assay (see, e.g., Molinaar (1996) Cell 86:391-399). A negative control may be included in these assays. By way of example, Wnt surrogate molecules may enhance β-catenin signaling by a factor of 2×, 5×, 10×, 100×, 1000×, 10000× or more as compared to the activity in the absence of the Wnt surrogate molecule when measured, for example when measured in the TOPFIash assay.

In certain embodiments, functional properties of the surrogate molecules may be assessed using a variety of methods known to the skilled person, including e.g., affinity/binding assays (for example, surface plasmon resonance, competitive inhibition assays), cytotoxicity assays, cell viability assays, cell proliferation or differentiation assays in response to the native molecule/ligand, cancer cell and/or tumor growth inhibition using in vitro or in vivo models, including but not limited to any described herein. The surrogate molecules may also be tested for effects on one or both co-receptor internalization, in vitro and in vivo efficacy, etc. Such assays may be performed using well-established protocols known to the skilled person (see e.g., Current Protocols in Molecular Biology (Greene Publ. Assoc. Inc. & John Wiley & Sons, Inc., NY, N.Y.); Current Protocols in Immunology (Edited by: John E. Coligan, Ada M. Kruisbeek, David H. Margulies, Ethan M. Shevach, Warren Strober 2001 John Wiley & Sons, NY, N.Y.); or commercially available kits.

In certain embodiments, a binding region of a surrogate molecule (e.g., an antigen-binding fragment of an anti-Fzd antibody) comprises one or more of the CDRs of the anti-co-receptor antibodies. In this regard, it has been shown in some cases that the transfer of only the VHCDR3 of an antibody can be performed while still retaining desired specific binding (Barbas et al., PNAS (1995) 92: 2529-2533). See also, McLane et al., PNAS (1995) 92:5214-5218, Barbas et al., J. Am. Chem. Soc. (1994) 116:2161-2162).

Also disclosed herein is a method for obtaining an antibody or antigen binding domain specific for a co-receptor, the method comprising providing by way of addition, deletion, substitution or insertion of one or more amino acids in the amino acid sequence of a VH domain set out herein or a VH domain which is an amino acid sequence variant of the VH domain, optionally combining the VH domain thus provided with one or more VL domains, and testing the VH domain or VH/VL combination or combinations to identify a specific binding member or an antibody antigen binding domain specific for one or more co-receptors and optionally with one or more desired properties. The VL domains may have an amino acid sequence which is substantially as set out herein. An analogous method may be employed in which one or more sequence variants of a VL domain disclosed herein are combined with one or more VH domains.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. See, generally, Davies et al. (1990) Annual Rev. Biochem. 59:439-473.

In certain embodiments, the surrogate molecules or binding regions thereof described herein have an affinity of less than about 10,000, less than about 1000, less than about 100, less than about 10, less than about 1 or less than about 0.1 nM, and in some embodiments, the antibodies may have even higher affinity for one or more co-receptors.

The constant regions of immunoglobulins show less sequence diversity than the variable regions, and are responsible for binding a number of natural proteins to elicit important biochemical events. In humans, there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region.

The Fc region of an antibody interacts with a number of Fc receptors and ligands, imparting an array of important functional capabilities referred to as effector functions. For IgG, the Fc region comprises Ig domains CH2 and CH3 and the N-terminal hinge leading into CH2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack.

The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). All FcγRs bind the same region on Fc, at the N-terminal end of the Cg2 (CH2) domain and the preceding hinge. This interaction is well characterized structurally (Sondermann et al., 2001, J Mol Biol 309:737-749), and several structures of the human Fc bound to the extracellular domain of human FcγRIIIb have been solved (pdb accession code 1E4K) (Sondermann et al., 2000, Nature 406:267-273.) (pdb accession codes 1IIS and 1IIX) (Radaev et al., 2001, J Biol Chem 276:16469-16477.)

The different IgG subclasses have different affinities for the FcγRs, with IgG1 and IgG3 typically binding substantially better to the receptors than IgG2 and IgG4 (Jefferis et al., 2002, Immunol Lett 82:57-65). All FcγRs bind the same region on IgG Fc, yet with different affinities: the high affinity binder FcγRI has a Kd for IgG1 of 10⁻⁸ M⁻¹, whereas the low affinity receptors FcγRII and FcγRIII generally bind at 10⁻⁶ and 10⁻⁵ respectively. The extracellular domains of FcγRIIIa and FcγRIIIb are 96% identical; however, FcγRIIIb does not have an intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. The receptors also differ in expression pattern and levels on different immune cells. Yet another level of complexity is the existence of a number of FcγR polymorphisms in the human proteome. A particularly relevant polymorphism with clinical significance is V158/F158 FcγRIIIa. Human IgG1 binds with greater affinity to the V158 allotype than to the F158 allotype. This difference in affinity, and presumably its effect on ADCC and/or ADCP, has been shown to be a significant determinant of the efficacy of the anti-CD20 antibody rituximab (Rituxan®, a registered trademark of IDEC Pharmaceuticals Corporation). Subjects with the V158 allotype respond favorably to rituximab treatment; however, subjects with the lower affinity F158 allotype respond poorly (Cartron et al., 2002, Blood 99:754-758). Approximately 10-20% of humans are V158/V158 homozygous, 45% are V158/F158 heterozygous, and 35-45% of humans are F158/F158 homozygous (Lehrnbecher et al., 1999, Blood 94:4220-4232; Cartron et al., 2002, Blood 99:754-758). Thus 80-90% of humans are poor responders, that is, they have at least one allele of the F158 FcγRIIIa.

The Fc region is also involved in activation of the complement cascade. In the classical complement pathway, C1 binds with its C1q subunits to Fc fragments of IgG or IgM, which has formed a complex with antigen(s). In certain embodiments of the invention, modifications to the Fc region comprise modifications that alter (either enhance or decrease) the ability of a Fzd-specific antibody as described herein to activate the complement system (see e.g., U.S. Pat. No. 7,740,847). To assess complement activation, a complement-dependent cytotoxicity (CDC) assay may be performed (See, e.g., Gazzano-Santoro et al., J. Immunol. Methods, 202:163 (1996)).

Thus in certain embodiments, the present invention provides the surrogate molecules having a modified Fc region with altered functional properties, such as reduced or enhanced CDC, ADCC, or ADCP activity, or enhanced binding affinity for a specific FcγR or increased serum half-life. Other modified Fc regions contemplated herein are described, for example, in issued U.S. Pat. Nos. 7,317,091; 7,657,380; 7,662,925; 6,538,124; 6,528,624; 7,297,775; 7,364,731; Published U.S. Applications US2009092599; US20080131435; US20080138344; and published International Applications WO2006/105338; WO2004/063351; WO2006/088494; WO2007/024249.

Structurally, the Fc region can be important for proper assembly of the msAb. In particular, modifications to the CH3 domain such as knobs-in-hole (see, e.g., WO1996/027011; and WO1998/050431) or Azymetric mutations (see, e.g., WO2012/58768) can prevent heavy chain mispairing. The present invention utilizes these mutations in certain Fc embodiments.

The surrogate molecules disclosed herein may also be modified to include an epitope tag or label, e.g., for use in purification or diagnostic applications. There are many linking groups known in the art for making antibody conjugates, including, for example, those disclosed in U.S. Pat. No. 5,208,020 or EP Patent 0 425 235 B1, and Chari et al., Cancer Research 52:

127-131 (1992). The linking groups include disulfide groups, thioether groups, acid labile groups, photolabile groups, peptidase labile groups, or esterase labile groups, as disclosed in the above-identified patents, disulfide and thioether groups being preferred.

In certain embodiments, and antigen-binding fragments thereof against one co-receptor and/or antibodies and antigen-binding fragments thereof against the other co-receptor present within a surrogate molecule are monoclonal. In certain embodiments, they are humanized.

The present invention further provides in certain embodiments an isolated nucleic acid encoding a polypeptide present in a surrogate molecule. Nucleic acids include DNA and RNA. These and related embodiments may include polynucleotides encoding antibody fragments that bind one or more co-receptors. The term “isolated polynucleotide” as used herein shall mean a polynucleotide of genomic, cDNA, or synthetic origin, or some combination thereof, which by virtue of its origin, the isolated polynucleotide: (1) is not associated with all or a portion of a polynucleotide in which the isolated polynucleotide is found in nature; (2) is linked to a polynucleotide to which it is not linked in nature, or (3) does not occur in nature as part of a larger sequence. An isolated

polynucleotide may include naturally occurring and/or artificial sequences.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

It will be appreciated by those of ordinary skill in the art that, as a result of the degeneracy of the genetic code, there are many nucleotide sequences that encodes an antibody as described herein. Some of these polynucleotides bear minimal sequence identity to the nucleotide sequence of the native or original polynucleotide sequence encoding a polypeptide within a surrogate molecule. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment of the invention, a mutagenesis approach, such as site-specific mutagenesis, may be employed for the preparation of variants and/or derivatives of the polypeptides described herein. By this approach, specific modifications in a polypeptide sequence can be made through mutagenesis of the underlying polynucleotides that encode them. These techniques provide a straightforward approach to prepare and test

sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the inventors contemplate the mutagenesis of the polynucleotide sequences that encode a polypeptide present in a surrogate molecule, to alter one or more properties of the encoded polypeptide, such as the binding affinity, or the function of a particular Fc region, or the affinity of the Fc region for a particular FcγR. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phages are readily commercially-available and their use is generally well-known to those skilled in the art. Double-stranded plasmids are also routinely employed in site directed mutagenesis that eliminates the step of transferring the gene of interest from a plasmid to a phage.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

In many embodiments, one or more nucleic acids encoding a polypeptide of a Wnt surrogate molecule are introduced directly into a host cell, and the cell incubated under conditions sufficient to induce expression of the encoded polypeptides. The surrogate polypeptides of this disclosure may be prepared using standard techniques well known to those of skill in the art in combination with the polypeptide and nucleic acid sequences provided herein. The polypeptide sequences may be used to determine appropriate nucleic acid sequences encoding the particular polypeptide disclosed thereby. The nucleic acid sequence may be optimized to reflect particular codon “preferences” for various expression systems according to standard methods well known to those of skill in the art.

According to certain related embodiments there is provided a recombinant host cell which comprises one or more constructs as described herein, e.g., a vector comprising a nucleic acid encoding a surrogate molecule or polypeptide thereof; and a method of production of the encoded product, which method comprises expression from encoding nucleic acid therefor. Expression may conveniently be achieved by culturing under appropriate conditions recombinant host cells containing the nucleic acid. Following production by expression, an antibody or antigen-binding fragment thereof, may be isolated and/or purified using any suitable technique, and then used as desired.

Polypeptides, and encoding nucleic acid molecules and vectors, may be isolated and/or purified, e.g. from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of polypeptides, e.g., antibodies and antigen-binding fragments thereof, in prokaryotic cells such as E. coli is well established in the art. For a review, see for example Pluckthun, A. Bio/Technology 9: 545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4: 573-576; Trill J. J. et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The present invention also provides, in certain embodiments, a method which comprises using a construct as stated above in an expression system in order to express a particular polypeptide such as a Wnt mimetic molecule. The term “transduction” is used to refer to the transfer of genes from one bacterium to another, usually by a phage.

Amino acid sequence modification(s) of any of the polypeptides described herein are contemplated. For example, it may be desirable to improve the binding affinity and/or other biological properties of the surrogate molecule. For example, amino acid sequence variants of a surrogate molecule may be prepared by introducing appropriate nucleotide changes into a polynucleotide that encodes the antibody, or a chain thereof, or by peptide synthesis. Such modifications include, for example, deletions from, and/or insertions into and/or substitutions of, residues within the amino acid sequences of the antibody. Any combination of deletion, insertion, and substitution may be made to arrive at the final surrogate molecule, provided that the final construct possesses the desired characteristics (e.g., high affinity binding to one or more co-receptors). The amino acid changes also may alter post-translational processes of the antibody, such as changing the number or position of glycosylation sites. Any of the variations and modifications described above for polypeptides of the present invention may be included in antibodies of the present invention.

The present disclosure provides variants of any of the polypeptides (e.g., surrogate molecules, or antibodies or antigen-binding fragments thereof) disclosed herein. In certain embodiments, a variant has at least 90%, at least 95%, at least 98%, or at least 99% identity to a polypeptide disclosed herein. In certain embodiments, such variant polypeptides bind to one or more first co-receptors, and/or to one or more second co-receptors, at least about 50%, at least about 70%, and in certain embodiments, at least about 90% as well as to a surrogate molecule specifically set forth herein. In further embodiments, such variant surrogate molecules bind to one or more first co-receptor, and/or to one or more second co-receptor, with greater affinity than the surrogate molecules set forth herein, for example, that bind quantitatively at least about 105%, 106%, 107%, 108%, 109%, or 110% as well as an antibody sequence specifically set forth herein.

In particular embodiments, the surrogate molecule or a binding region thereof, e.g., a Fab, scFv, or VHH may comprise: a) a heavy chain variable region comprising: i. a CDR1 region that is identical in amino acid sequence to the heavy chain CDR1 region of a selected antibody described herein; ii. a CDR2 region that is identical in amino acid sequence to the heavy chain CDR2 region of the selected antibody; and iii. a CDR3 region that is identical in amino acid sequence to the heavy chain CDR3 region of the selected antibody; and/or b) a light chain variable domain comprising: i. a CDR1 region that is identical in amino acid sequence to the light chain CDR1 region of the selected antibody; ii. a CDR2 region that is identical in amino acid sequence to the light chain CDR2 region of the selected antibody; and iii. a CDR3 region that is identical in amino acid sequence to the light chain CDR3 region of the selected antibody; wherein the antibody specifically binds a selected target. In a further embodiment, the antibody, or antigen-binding fragment thereof, is a variant antibody or antigen-binding fragment thereof wherein the variant comprises a heavy and light chain identical to the selected antibody except for up to 8, 9, 10, 11, 12, 13, 14, 15, or more amino acid substitutions in the CDR regions of the VH and VL regions. In this regard, there may be 1, 2, 3, 4, 5, 6, 7, 8, or in certain embodiments, 9, 10, 11, 12, 13, 14, 15 more amino acid substitutions in the CDR regions of the selected antibody. Substitutions may be in CDRs either in the VH and/or the VL regions. (See e.g., Muller, 1998, Structure 6:1153-1167).

In particular embodiments, the surrogate molecule or a binding region thereof, e.g., a Fab, scFv, or VHH/sdAb, may have: a) a heavy chain variable region having an amino acid sequence that is at least 80% identical, at least 95% identical, at least 90%, at least 95% or at least 98% or 99% identical, to the heavy chain variable region of an antibody or antigen-binding fragments thereof described herein; and/or b) a light chain variable region having an amino acid sequence that is at least 80% identical, at least 85%, at least 90%, at least 95% or at least 98% or 99% identical, to the light chain variable region of an antibody or antigen-binding fragments thereof described herein.

A polypeptide has a certain percent “sequence identity” to another polypeptide, meaning that, when aligned, that percentage of amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

In particular embodiments, the surrogate molecule or a binding region thereof, e.g., a Fab, scFv, or VHH may comprise: a) a heavy chain variable region comprising: i. a CDR1 region that is identical in amino acid sequence to the heavy chain CDR1 region of a selected antibody described herein; ii. a CDR2 region that is identical in amino acid sequence to the heavy chain CDR2 region of the selected antibody; and iii. a CDR3 region that is identical in amino acid sequence to the heavy chain CDR3 region of the selected antibody; and b) a light chain variable domain comprising: i. a CDR1 region that is identical in amino acid sequence to the light chain CDR1 region of the selected antibody; ii. a CDR2 region that is identical in amino acid sequence to the light chain CDR2 region of the selected antibody; and iii. a CDR3 region that is identical in amino acid sequence to the light chain CDR3 region of the selected antibody; wherein the antibody specifically binds a selected target (e.g., a Fzd receptor, such as Fzd1). In a further embodiment, the antibody, or antigen-binding fragment thereof, is a variant antibody wherein the variant comprises a heavy and light chain identical to the selected antibody except for up to 8, 9, 10, 11, 12, 13, 14, 15, or more amino acid substitutions in the CDR regions of the VH and VL regions. In this regard, there may be 1, 2, 3, 4, 5, 6, 7, 8, or in certain embodiments, 9, 10, 11, 12, 13, 14, 15 more amino acid substitutions in the CDR regions of the selected antibody. Substitutions may be in CDRs either in the VH and/or the VL regions. (See e.g., Muller, 1998, Structure 6:1153-1167).

Determination of the three-dimensional structures of representative polypeptides (e.g., variant Fzd binding regions or LRP5/6 binding regions of Wnt surrogate molecules as provided herein) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, such as for rational design of binding regions include VIVID which is a molecular visualization program for displaying, animating, and analyzing large biomolecular systems using 3-D graphics and built-in scripting (see the website for the Theoretical and Computational Biophysics Group, University of Illinois at Urbana-Champagne, at ks.uiuc.edu/Research/vmd/. Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106: 765), which assess force field calculations, and analysis (see also, Eisenfield et al. (1991) Am. J. Physiol. 261:C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J.

Biomol. Struct. Dyn. 9:475-488). A variety of appropriate computational computer programs are also commercially available, such as from Schrödinger (Munich, Germany).

### IV. Compositions

Pharmaceutical compositions comprising a surrogate molecule described herein and one or more pharmaceutically acceptable diluent, carrier, or excipient are also disclosed.

In further embodiments, pharmaceutical compositions comprising a polynucleotide comprising a nucleic acid sequence encoding a surrogate molecule described herein and one or more pharmaceutically acceptable diluent, carrier, or excipient are also disclosed. In particular embodiments, the pharmaceutical composition further comprises one or more polynucleotides comprising a nucleic acid sequence encoding a naturally occurring co-receptor ligand polypeptide. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a polyA tail. In other embodiments, the polynucleotides are expression cassettes comprising a promoter operatively linked to the coding sequences. In certain embodiments, the nucleic acid sequence encoding the surrogate molecule and the nucleic acid sequence encoding naturally occurring co-receptor ligand polypeptide are present in the same polynucleotide.

In further embodiments, pharmaceutical compositions comprising an expression vector, e.g., a viral vector, comprising a polynucleotide comprising a nucleic acid sequence encoding a surrogate molecule described herein and one or more pharmaceutically acceptable diluent, carrier, or excipient are also disclosed. In particular embodiments, the pharmaceutical composition further comprises an expression vector, e.g., a viral vector, comprising a polynucleotide comprising a nucleic acid sequence encoding a naturally occurring co-receptor ligand polypeptide. In certain embodiments, the nucleic acid sequence encoding the surrogate molecule and the nucleic acid sequence encoding the naturally occurring co-receptor ligand polypeptide are present in the same polynucleotide, e.g., expression cassette.

The present invention further contemplates a pharmaceutical composition comprising a cell comprising an expression vector comprising a polynucleotide comprising a promoter operatively linked to a nucleic acid encoding a surrogate molecule and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a polypeptide corresponding to the natural ligand of the receptors. In particular embodiments, the cell is a heterologous cell or an autologous cell obtained from the subject to be treated. In particular embodiments, the cell is a stem cell, e.g., an adipose-derived stem cell or a hematopoietic stem cell.

The subject molecules, alone or in combination, can be combined with pharmaceutically-acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the surrogate molecule (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms are dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g. syringe, e.g. a prefilled syringe, together with instructions for administration.

The pharmaceutical compositions of the invention encompass any pharmaceutically acceptable salts, esters, or salts of such esters, or any other compound which, upon administration to an animal comprising a human, is capable of providing (directly or indirectly) the biologically active antibody or antigen-binding fragment thereof.

The present invention includes pharmaceutically acceptable salts of a Wnt surrogate molecule described herein. The term “pharmaceutically acceptable salt” refers to physiologically and pharmaceutically acceptable salts of the compounds of the invention: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see “Handbook of Pharmaceutical Salts: Properties, Selection, and Use” by Stahl and Wermuth (Wiley-VCH, 2002).

Pharmaceutically acceptable base addition salts are formed with metals or amines, such as alkali and alkaline earth metals or organic amines. Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

In some embodiments, the pharmaceutical composition provided herein comprise a therapeutically effective amount of a Wnt surrogate molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

### V. Methods of Use

For illustrative purposes only, the surrogate molecule is a Wnt mimetic and can be used as to treat various diseases or disorders where tissue regeneration is necessary. Such diseases include, but are not limited to: increase bone growth or regeneration, bone grafting, healing of bone fractures, treatment of osteoporosis and osteoporotic fractures, vertebral compression fractures, spinal fusion, osseointegration of orthopedic devices, tendon-bone integration, tooth growth and regeneration, dental implantation, periodontal diseases, maxillofacial reconstruction, and osteonecrosis of the jaw. Also contemplated are: treatment of alopecia; enhancing regeneration of sensory organs, e.g. treatment of hearing loss, including internal and external auditory hair cells, treatment of vestibular hypofunction, treatment of macular degeneration, treatment of vitreoretinopathy, diabetic retinopathy, other diseases of retinal degeneration, Fuchs' dystrophy, other cornea disease, etc.; treatment of stroke, traumatic brain injury, Alzheimer's disease, multiple sclerosis and other conditions affecting the blood brain barrier; treatment of spinal cord injuries, other spinal cord diseases. The compositions of this invention may also be used in treatment of oral mucositis, treatment of short bowel syndrome, inflammatory bowel diseases (IBD), other gastrointestinal disorders; treatment of metabolic syndrome, dyslipidemia, treatment of diabetes, treatment of pancreatitis, conditions where exocrine or endocrine pancreas tissues are damaged; conditions where enhanced epidermal regeneration is desired, e.g., epidermal wound healing, treatment of diabetic foot ulcers, syndromes involving tooth, nail, or dermal hypoplasia, etc., conditions where angiogenesis is beneficial; treatment of myocardial infarction, coronary artery disease, heart failure; enhanced growth of hematopoietic cells, e.g. enhancement of hematopoietic stem cell transplants from bone marrow, mobilized peripheral blood, treatment of immunodeficiencies, graft versus host diseases, etc.; treatment of acute kidney injuries, chronic kidney diseases; treatment of lung diseases, chronic obstructive pulmonary diseases (COPD), idiopathic pulmonary fibrosis (IPF) enhanced regeneration of lung tissues. The compositions of the present invention may also be used in enhanced regeneration of liver cells, e.g. liver regeneration, treatment of cirrhosis, enhancement of liver transplantations, treatment of acute liver failure, treatment of chronic liver diseases with hepatitis C or B virus infection or post-antiviral drug therapies, alcoholic liver diseases, alcoholic hepatitis, non-alcoholic liver diseases with steatosis or steatohepatitis, and the like. The compositions of this invention may treat diseases and disorders including, without limitation, conditions in which regenerative cell growth is desired.

In particular embodiments, a pharmaceutical composition is administered parenterally, e.g., intravenously, orally, rectally, or by injection. In some embodiments, it is administered locally, e.g., topically or intramuscularly. In some embodiments, a composition is administered to target tissues, e.g., to bone, joints, ear tissue, eye tissue, gastrointestinal tract, skin, a wound site or spinal cord. Methods of the invention may be practiced in vivo or ex vivo. In some embodiments, the contacting of a target cell or tissue with a surrogate molecule is performed ex vivo, with subsequent implantation of the cells or tissues, e.g., activated stem or progenitor cells, into the subject. The skilled artisan can determine an appropriate site of and route of administration based on the disease or disorder being treated.

The dose and dosage regimen may depend upon a variety of factors readily determined by a physician, such as the nature of the disease or disorder, the characteristics of the subject, and the subject's history. In particular embodiments, the amount of a surrogate molecule administered or provided to the subject is in the range of about 0.01 mg/kg to about 50 mg/kg, 0.1 mg/kg to about 500 mg/kg, or about 0.1 mg/kg to about 50 mg/kg of the subject's body weight.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

### EXAMPLES

Described below are three formats in which two Fab domains from different antibodies were connected on both light- and heavy-chains, via various types of linkers, to result in a tetravalent bispecific molecule. Such bispecific molecules have general application in bringing two different host antigens together with avidity due to their ability to engage antigens bivalently and modulate various biological pathways. In these formats (FIGS. 1A-1C) which allows production of tetravalent bispecific molecules using only two plasmids, one each for light- and heavy-chains, the first (outer) Fab-domain is connected to the N-terminus the second (inner) Fab-domain, which in turn is connected to the N-terminus of the Fc-domain. Three different ways in which such molecules could be designed are termed as dual-Fab-Ig (DFab-Ig without any cross-over), DFab-Ig in which outer Fab's light- and heavy-chains are crossed with respect to inner Fab (DFabOC-Ig), and DFab-Ig in which inner Fab's light- and heavy-chains are crossed with respect to the Fc domain (DFabIC-Ig). Here we fused antibodies against Fzd and Lrp5/6 receptors to illustrate applicability of DFab-Ig, DFabOC-Ig, and DFabIC-Ig formats as both agonists and antagonists of Wnt-signaling.

### Example 1

### Materials and Methods

Molecular biology methods: Synthetic DNA-fragments expressing light-chain and heavy-chain WNT-agonist molecules in DFab-Ig, DFabOC-Ig, DFabIC-Ig, and CROSS_FIT formats made-up of a Frizzled antibodies (1RC07, 18R5 (Gurney et al., 2012)) and a Lrp5/6 antibodies (10SA7, YW211.31.57 (U.S. Pat. No. 8,846,041)), and WNT-antogonist molecules in DFabOC-Ig format made-up of the Frizzled antibody 18R5 and PD-1 antibody pembrolizumab (pembro) were cloned into pcDNA3.1(+)-based plasmid containing secretion signal-peptide from Kappa-light-chain (MDMRVPAQLLGLLLLWLRGARC) (SEQ ID NO:40) using Gibson Assembly method following standard protocols. In the case of heavy-chain plasmids either CH1 or CLkappa domains were fused to the N-terminus of a human IgG1 Fc domain containing L234A/L235A/P329G mutations (LALAPG) (SEQ ID NO:41) to eliminate effector function (Lo et al., 2017). Amino acid sequences for all designed light- and heavy-chain plasmids are listed in Table 1.

TABLE 1
SEQ ID NOs and Description of constructs designed for DFab-Ig (no cross over),
DFabOC-Ig (“outer-Fab” cross over), DFabIC-Ig (“inner Fab” cross over), and CrossFIT-Ig
tetravalent-bispecific molecules. Linker residues are highlighted with gray-background and
sequence of the variable domains are shown in italics.

| | | | |
|---|---|---|---|
| SEQ | Clone | | |
| ID | ID | | |
| NO | number | Description | Amino acid sequence |

Amino acid sequence for the constructs used for Wnt agonists as DFab-Ig (no cross-over)

| | | | |
|---|---|---|---|
| 1 | 13017 | 10SA7-VH_hinge- | EVQLVESGGGLVQPGGSLRLSCAASGFTFSSYAMHW |
| | | 10mer-hinge_1RC07- | VRQAPGKGLEWVASISSTSGSKYYADSVKGRFTISRDN |
| | | VH | SKNTLYLQMNSLRAEDTAVYSCAKTYYDFWSGYYTFD |
| | | (hinge-10mer-hinge = | YWGQGTLVTVSSASTKGPSVFPLAPSSKSTSGGTAA |
| | | HFL2) | LGCLVKDYFPEPVTVSWNSGALTSGVHTFPAVLQ |
| | | | SSGLYSLSSVVTVPSSSLGTQTYICNVNHKPSNTK |
| | | | 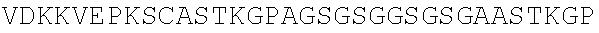 |
| | | | QVQLQQWGAGLLKPSETLSLTCAVSGASFSGHYWTW |
| | | | IRQPPGKGLEWIGEIDHTGSTNYEPSLRSRVTISVDTS |
| | | | KNQFSLNLKSVTAADTAVYYCARGGQGGYDWGHYH |
| | | | GLDVWGQGTTVTVSSASTKGPSVFPLAPSSKSTSGG |
| | | | TAALGCLVKDYFPEPVTVSWNSGALTSGVHTFPA |
| | | | VLQSSGLYSLSSVVTVPSSSLGTQTYICNVNHKPS |
| | | | NTKVDKKVEPKSCDKTHTCPPCPAPEAAGGPSVF |
| | | | LFPPKPKDTLMISRTPEVTCVVVDVSHEDPEVKFN |
| | | | WYVDGVEVHNAKTKPREEQYNSTYRVVSVLTVL |
| | | | HQDWLNGKEYKCKVSNKALGAPIEKTISKAKGQP |
| | | | REPQVYTLPPSREEMTKNQVSLTCLVKGFYPSDIA |
| | | | VEWESNGQPENNYKTTPPVLDSDGSFFLYSKLTV |
| | | | DKSRWQQGNVFSCSVMHEALHNHYTQKSLSLSP |
| | | | GK* |
| 2 | 13018 | 10SA7-VL-hk_hinge- | DIQMTQSPSSLSASVGDRVTITCRASQSISSYLNWYQQ |
| | | 10mer- | KPGKAPKLLIYAASSLQSGVPSRFSGSGSGTDFTLTISS |
| | | hinge_1RC07_VL | LQPEDFATYYCQQSYSTPLTFGGGTKVEIKRTVAAPS |
| | | (hinge-10mer-hinge = | VFIFPPSDEQLKSGTASVVCLLNNFYPREAKVQWK |
| | | HFL2) | VDNALQSGNSQESVTEQDSKDSTYSLSSTLTLSKA |
| | | | 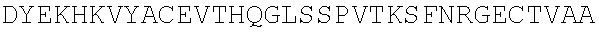 |
| | | | 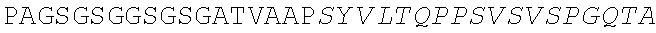 |
| | | | SITCSGDKVGHKYASWYQQKPGQSPVLVIYEDSQRPS |
| | | | GIPVRFSGSNSGNTATLTISGTQAMDEADYYCQAWDS |
| | | | STDVVFGGGTKLTVLGQPKAAPSVTLFPPSSEELQA |
| | | | NKATLVCLISDFYPGAVTVAWKADSSPVKAGVET |
| | | | TTPSKQSNNKYAASSYLSLTPEQWKSHRSYSCQV |
| | | | THEGSTVEKTVAPTECS* |
| 3 | 13019 | 1RC07-VH_hinge- | QVQLQQWGAGLLKPSETLSLTCAVSGASFSGHYWTW |
| | | 10mer-hinge_ | IRQPPGKGLEWIGEIDHTGSTNYEPSLRSRVTISVDTS |
| | | 10SA7_VH | KNQFSLNLKSVTAADTAVYYCARGGQGGYDWGHYH |
| | | (hinge-10mer-hinge = | GLDVWGQGTTVTVSSASTKGPSVFPLAPSSKSTSGG |
| | | HFL2) | TAALGCLVKDYFPEPVTVSWNSGALTSGVHTFPA |
| | | | VLQSSGLYSLSSVVTVPSSSLGTQTYICNVNHKPS |
| | | | 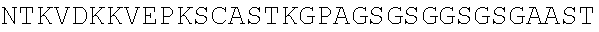 |
| | | | 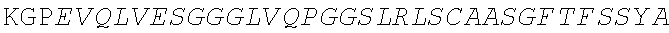 |
| | | | MHWVRQAPGKGLEWVASISSTSGSKYYADSVKGRFTI |
| | | | SRDNSKNTLYLQMNSLRAEDTAVYSCAKTYYDFWSGY |
| | | | YTFDYWGQGTLVTVSSASTKGPSVFPLAPSSKSTSG |
| | | | GTAALGCLVKDYFPEPVTVSWNSGALTSGVHTFP |
| | | | AVLQSSGLYSLSSVVTVPSSSLGTQTYICNVNHKP |
| | | | SNTKVDKKVEPKSCDKTHTCPPCPAPEAAGGPSV |
| | | | FLFPPKPKDTLMISRTPEVTCVVVDVSHEDPEVKF |
| | | | NWYVDGVEVHNAKTKPREEQYNSTYRVVSVLTV |
| | | | LHQDWLNGKEYKCKVSNKALGAPIEKTISKAKGQ |
| | | | PREPQVYTLPPSREEMTKNQVSLTCLVKGFYPSDI |
| | | | AVEWESNGQPENNYKTTPPVLDSDGSFFLYSKLT |
| | | | VDKSRWQQGNVFSCSVMHEALHNHYTQKSLSLS |
| | | | PGK* |
| 4 | 13020 | 1RC07-VL-hinge- | SYVLTQPPSVSVSPGQTASITCSGDKVGHKYASWYQQ |
| | | 10mer-hinge_10SA7- | KPGQSPVLVIYEDSQRPSGIPVRFSGSNSGNTATLTIS |
| | | VL | GTQAMDEADYYCQAWDSSTDVVFGGGTKLTVLGQP |
| | | (hinge-10mer-hinge = | KAAPSVTLFPPSSEELQANKATLVCLISDFYPGAV |
| | | HFL2) | TVAWKADSSPVKAGVETTTPSKQSNNKYAASSYL |
| | | | 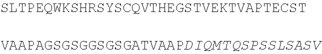 |
| | | | GDRVTITCRASQSISSYLNWYQQKPGKAPKLLIYAASS |
| | | | LQSGVPSRFSGSGSGTDFTLTISSLQPEDFATYYCQQS |
| | | | YSTPLTFGGGTKVEIKRTVAAPSVFIFPPSDEQLKSG |
| | | | TASVVCLLNNFYPREAKVQWKVDNALQSGNSQE |
| | | | SVTEQDSKDSTYSLSSTLTLSKADYEKHKVYACE |
| | | | VTHQGLSSPVTKSFNRGEC* |
| 5 | 13021 | 10SA7-VH_hinge- | EVQLVESGGGLVQPGGSLRLSCAASGFTFSSYAMHW |
| | | helix1- | VRQAPGKGLEWVASISSTSGSKYYADSVKGRFTISRDN |
| | | hinge_1RC07_VH | SKNTLYLQMNSLRAEDTAVYSCAKTYYDFWSGYYTFD |
| | | (hinge-helix1-hinge = | YWGQGTLVTVSSASTKGPSVFPLAPSSKSTSGGTAA |
| | | HHL1) | LGCLVKDYFPEPVTVSWNSGALTSGVHTFPAVLQ |
| | | | SSGLYSLSSVVTVPSSSLGTQTYICNVNHKPSNTK |
| | | | 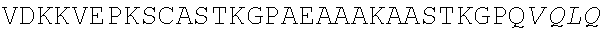 |
| | | | QWGAGLLKPSETLSLTCAVSGASFSGHYWTWIRQPP |
| | | | GKGLEWIGEIDHTGSTNYEPSLRSRVTISVDTSKNQFS |
| | | | LNLKSVTAADTAVYYCARGGQGGYDWGHYHGLDVW |
| | | | GQGTTVTVSSASTKGPSVFPLAPSSKSTSGGTAALG |
| | | | CLVKDYFPEPVTVSWNSGALTSGVHTFPAVLQSS |
| | | | GLYSLSSVVTVPSSSLGTQTYICNVNHKPSNTKVD |
| | | | KKVEPKSCDKTHTCPPCPAPEAAGGPSVFLFPPKP |
| | | | KDTLMISRTPEVTCVVVDVSHEDPEVKFNWYVDG |
| | | | VEVHNAKTKPREEQYNSTYRVVSVLTVLHQDWL |
| | | | NGKEYKCKVSNKALGAPIEKTISKAKGQPREPQV |
| | | | YTLPPSREEMTKNQVSLTCLVKGFYPSDIAVEWES |
| | | | NGQPENNYKTTPPVLDSDGSFFLYSKLTVDKSRW |
| | | | QQGNVFSCSVMHEALHNHYTQKSLSLSPGK* |
| 6 | 13022 | 10SA7-VL_hinge- | DIQMTQSPSSLSASVGDRVTITCRASQSISSYLNWYQQ |
| | | helix1-hinge_1RC07- | KPGKAPKLLIYAASSLQSGVPSRFSGSGSGTDFTLTISS |
| | | VL | LQPEDFATYYCQQSYSTPLTFGGGTKVEIKRTVAAPS |
| | | (hinge-helix1-hinge = | VFIFPPSDEQLKSGTASVVCLLNNFYPREAKVQWK |
| | | HHL1) | VDNALQSGNSQESVTEQDSKDSTYSLSSTLTLSKA |
| | | | 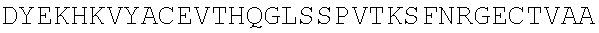 |
| | | | 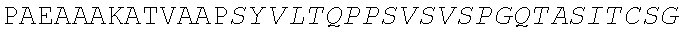 |
| | | | DKVGHKYASWYQQKPGQSPVLVIYEDSQRPSGIPVR |
| | | | FSGSNSGNTATLTISGTQAMDEADYYCQAWDSSTDVV |
| | | | FGGGTKLTVLGQPKAAPSVTLFPPSSEELQANKAT |
| | | | LVCLISDFYPGAVTVAWKADSSPVKAGVETTTPS |
| | | | KQSNNKYAASSYLSLTPEQWKSHRSYSCQVTHEG |
| | | | STVEKTVAPTECS* |
| 7 | 13023 | 1RC07-VH_hinge- | QVQLQQWGAGLLKPSETLSLTCAVSGASFSGHYWTW |
| | | helix1-hinge_10SA7- | IRQPPGKGLEWIGEIDHTGSTNYEPSLRSRVTISVDTS |
| | | VH | KNQFSLNLKSVTAADTAVYYCARGGQGGYDWGHYH |
| | | (hinge-helix1-hinge = | GLDVWGQGTTVTVSSASTKGPSVFPLAPSSKSTSGG |
| | | HHL1) | TAALGCLVKDYFPEPVTVSWNSGALTSGVHTFPA |
| | | | VLQSSGLYSLSSVVTVPSSSLGTQTYICNVNHKPS |
| | | | 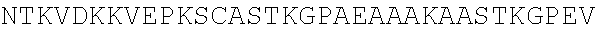 |
| | | | QLVESGGGLVQPGGSLRLSCAASGFTFSSYAMHW |
| | | | VRQAPGKGLEWVASISSTSGSKYYADSVKGRFTIS |
| | | | RDNSKNTLYLQMNSLRAEDTAVYSCAKTYYDFW |
| | | | SGYYTFDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCDKTHTCPPCPAPEAAGG |
| | | | PSVFLFPPKPKDTLMISRTPEVTCVVVDVSHEDPE |
| | | | VKFNWYVDGVEVHNAKTKPREEQYNSTYRVVSV |
| | | | LTVLHQDWLNGKEYKCKVSNKALGAPIEKTISKA |
| | | | KGQPREPQVYTLPPSREEMTKNQVSLTCLVKGFY |
| | | | PSDIAVEWESNGQPENNYKTTPPVLDSDGSFFLYS |
| | | | KLTVDKSRWQQGNVFSCSVMHEALHNHYTQKSL |
| | | | SLSPGK* |
| 8 | 13024 | 1RC07-VL_ hinge- | SYVLTQPPSVSVSPGQTASITCSGDKVGHKYASWYQQ |
| | | helix1-hinge_10SA7- | KPGQSPVLVIYEDSQRPSGIPVRFSGSNSGNTATLTIS |
| | | VL | GTQAMDEADYYCQAWDSSTDVVFGGGTKLTVLGQP |
| | | (hinge-helix1-hinge = | KAAPSVTLFPPSSEELQANKATLVCLISDFYPGAV |
| | | HHL1) | TVAWKADSSPVKAGVETTTPSKQSNNKYAASSYL |
| | | | 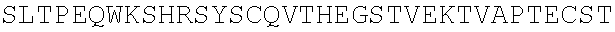 |
| | | | 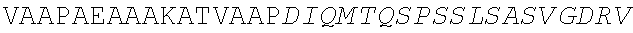 |
| | | | TITCRASQSISSYLNWYQQKPGKAPKLLIYAASSLQSG |
| | | | VPSRFSGSGSGTDFTLTISSLQPEDFATYYCQQSYSTP |
| | | | LTFGGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVTH |
| | | | QGLSSPVTKSFNRGEC* |
| 9 | 13025 | 10SA7-VH_hinge- | EVQLVESGGGLVQPGGSLRLSCAASGFTFSSYAMHW |
| | | helix2-hinge_1RC07- | VRQAPGKGLEWVASISSTSGSKYYADSVKGRFTISRDN |
| | | VH | SKNTLYLQMNSLRAEDTAVYSCAKTYYDFWSGYYTFD |
| | | (hinge-helix1-hinge = | YWGQGTLVTVSSASTKGPSVFPLAPSSKSTSGGTAA |
| | | HHL2) | LGCLVKDYFPEPVTVSWNSGALTSGVHTFPAVLQ |
| | | | SSGLYSLSSVVTVPSSSLGTQTYICNVNHKPSNTK |
| | | | 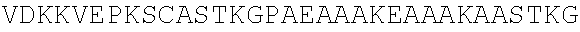 |
| | | | 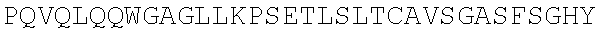 |
| | | | WTWIRQPPGKGLEWIGEIDHTGSTNYEPSLRSRVT |
| | | | ISVDTSKNQFSLNLKSVTAADTAVYYCARGGQGG |
| | | | YDWGHYHGLDVWGQGTTVTVSSASTKGPSVFPL |
| | | | APSSKSTSGGTAALGCLVKDYFPEPVTVSWNSGA |
| | | | LTSGVHTFPAVLQSSGLYSLSSVVTVPSSSLGTQT |
| | | | YICNVNHKPSNTKVDKKVEPKSCDKTHTCPPCPA |
| | | | PEAAGGPSVFLFPPKPKDTLMISRTPEVTCVVVDV |
| | | | SHEDPEVKFNWYVDGVEVHNAKTKPREEQYNST |
| | | | YRVVSVLTVLHQDWLNGKEYKCKVSNKALGAPI |
| | | | EKTISKAKGQPREPQVYTLPPSREEMTKNQVSLTC |
| | | | LVKGFYPSDIAVEWESNGQPENNYKTTPPVLDSD |
| | | | GSFFLYSKLTVDKSRWQQGNVFSCSVMHEALHN |
| | | | HYTQKSLSLSPGK* |
| 10 | 13026 | 10SA7-VL_hinge- | DIQMTQSPSSLSASVGDRVTITCRASQSISSYLNWYQQ |
| | | helix2-hinge_1RC07- | KPGKAPKLLIYAASSLQSGVPSRFSGSGSGTDFTLTISS |
| | | VL | LQPEDFATYYCQQSYSTPLTFGGGTKVEIKRTVAAPS |
| | | (hinge-helix2-hinge = | VFIFPPSDEQLKSGTASVVCLLNNFYPREAKVQWK |
| | | HHL2) | VDNALQSGNSQESVTEQDSKDSTYSLSSTLTLSKA |
| | | | 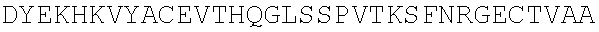 |
| | | | 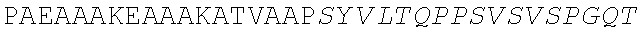 |
| | | | ASITCSGDKVGHKYASWYQQKPGQSPVLVIYEDSQRP |
| | | | SGIPVRFSGSNSGNTATLTISGTQAMDEADYYCQAWD |
| | | | SSTDVVFGGGTKLTVLGQPKAAPSVTLFPPSSEELQ |
| | | | ANKATLVCLISDFYPGAVTVAWKADSSPVKAGVE |
| | | | TTTPSKQSNNKYAASSYLSLTPEQWKSHRSYSCQ |
| | | | VTHEGSTVEKTVAPTECS* |
| 11 | 13027 | 1RC07-VH_hinge- | QVQLQQWGAGLLKPSETLSLTCAVSGASFSGHYWTW |
| | | helix2-hinge_10SA7- | IRQPPGKGLEWIGEIDHTGSTNYEPSLRSRVTISVDTS |
| | | VH | KNQFSLNLKSVTAADTAVYYCARGGQGGYDWGHYH |
| | | (hinge-helix2-hinge = | GLDVWGQGTTVTVSSASTKGPSVFPLAPSSKSTSGG |
| | | HHL2) | TAALGCLVKDYFPEPVTVSWNSGALTSGVHTFPA |
| | | | VLQSSGLYSLSSVVTVPSSSLGTQTYICNVNHKPS |
| | | | 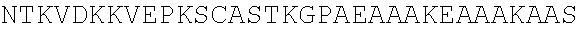 |
| | | | 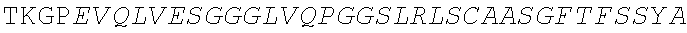 |
| | | | MHWVRQAPGKGLEWVASISSTSGSKYYADSVKGRFTI |
| | | | SRDNSKNTLYLQMNSLRAEDTAVYSCAKTYYDFWSGY |
| | | | YTFDYWGQGTLVTVSSASTKGPSVFPLAPSSKSTSG |
| | | | GTAALGCLVKDYFPEPVTVSWNSGALTSGVHTFP |
| | | | AVLQSSGLYSLSSVVTVPSSSLGTQTYICNVNHKP |
| | | | SNTKVDKKVEPKSCDKTHTCPPCPAPEAAGGPSV |
| | | | FLFPPKPKDTLMISRTPEVTCVVVDVSHEDPEVKF |
| | | | NWYVDGVEVHNAKTKPREEQYNSTYRVVSVLTV |
| | | | LHQDWLNGKEYKCKVSNKALGAPIEKTISKAKGQ |
| | | | PREPQVYTLPPSREEMTKNQVSLTCLVKGFYPSDI |
| | | | AVEWESNGQPENNYKTTPPVLDSDGSFFLYSKLT |
| | | | VDKSRWQQGNVFSCSVMHEALHNHYTQKSLSLS |
| | | | PGK |
| 12 | 13028 | 1RC07-VL_hinge- | SYVLTQPPSVSVSPGQTASITCSGDKVGHKYASWYQQ |
| | | helix2-hinge_10SA7- | KPGQSPVLVIYEDSQRPSGIPVRFSGSNSGNTATLTIS |
| | | VL | GTQAMDEADYYCQAWDSSTDVVFGGGTKLTVLGQP |
| | | (hinge-helix2-hinge = | KAAPSVTLFPPSSEELQANKATLVCLISDFYPGAV |
| | | HHL2) | TVAWKADSSPVKAGVETTTPSKQSNNKYAASSYL |
| | | | 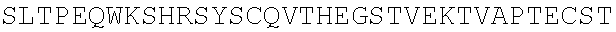 |
| | | | 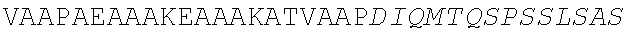 |
| | | | VGDRVTITCRASQSISSYLNWYQQKPGKAPKLLIYAAS |
| | | | SLQSGVPSRFSGSGSGTDFTLTISSLQPEDFATYYCQQ |
| | | | SYSTPLTFGGGTKVEIKRTVAAPSVFIFPPSDEQLKS |
| | | | GTASVVCLLNNFYPREAKVQWKVDNALQSGNSQ |
| | | | ESVTEQDSKDSTYSLSSTLTLSKADYEKHKVYACE |
| | | | VTHQGLSSPVTKSFNRGEC* |
| 13 | 13029 | 10SA7-VH_hinge- | EVQLVESGGGLVQPGGSLRLSCAASGFTFSSYAMHW |
| | | helix3-hinge_1RC07- | VRQAPGKGLEWVASISSTSGSKYYADSVKGRFTISRDN |
| | | VH | SKNTLYLQMNSLRAEDTAVYSCAKTYYDFWSGYYTFD |
| | | (hinge-helix3-hinge = | YWGQGTLVTVSSASTKGPSVFPLAPSSKSTSGGTAA |
| | | HHL3) | LGCLVKDYFPEPVTVSWNSGALTSGVHTFPAVLQ |
| | | | SSGLYSLSSVVTVPSSSLGTQTYICNVNHKPSNTK |
| | | | 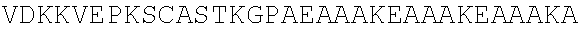 |
| | | | 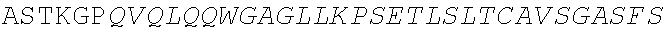 |
| | | | GHYWTWIRQPPGKGLEWIGEIDHTGSTNYEPSLRSRV |
| | | | TISVDTSKNQFSLNLKSVTAADTAVYYCARGGQGGYD |
| | | | WGHYHGLDVWGQGTTVTVSSASTKGPSVFPLAPSS |
| | | | KSTSGGTAALGCLVKDYFPEPVTVSWNSGALTSG |
| | | | VHTFPAVLQSSGLYSLSSVVTVPSSSLGTQTYICNV |
| | | | NHKPSNTKVDKKVEPKSCDKTHTCPPCPAPEAAG |
| | | | GPSVFLFPPKPKD TLMISRTPEVTCVVVDVSHEDP |
| | | | EVKFNWYVDGVEVHNAKTKPREEQYNSTYRVVS |
| | | | VLTVLHQDWLNGKEYKCKVSNKALGAPIEKTISK |
| | | | AKGQPREPQVYTLPPSREEMTKNQVSLTCLVKGF |
| | | | YPSDIAVEWESNGQPENNYKTTPPVLDSDGSFFLY |
| | | | SKLTVDKSRWQQGNVFSCSVMHEALHNHYTQKS |
| | | | LSLSPGK* |
| 14 | 13030 | 10SA7-VL_hinge- | DIQMTQSPSSLSASVGDRVTITCRASQSISSYLNWYQQ |
| | | helix3-hinge_1RC07- | KPGKAPKLLIYAASSLQSGVPSRFSGSGSGTDFTLTISS |
| | | VL | LQPEDFATYYCQQSYSTPLTFGGGTKVEIKRTVAAPS |
| | | (hinge-helix3-hinge = | VFIFPPSDEQLKSGTASVVCLLNNFYPREAKVQWK |
| | | HHL3) | VDNALQSGNSQESVTEQDSKDSTYSLSSTLTLSKA |
| | | | 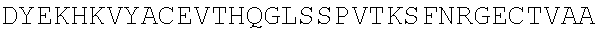 |
| | | | 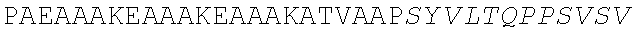 |
| | | | SPGQTASITCSGDKVGHKYASWYQQKPGQSPVLVIYE |
| | | | DSQRPSGIPVRFSGSNSGNTATLTISGTQAMDEADYY |
| | | | CQAWDSSTDVVFGGGTKLTVLGQPKAAPSVTLFPPS |
| | | | SEELQANKATLVCLISDFYPGAVTVAWKADSSPV |
| | | | KAGVETTTPSKQSNNKYAASSYLSLTPEQWKSHR |
| | | | SYSCQVTHEGSTVEKTVAPTECS* |
| 15 | 13031 | 1RC07-VH_hinge- | QVQLQQWGAGLLKPSETLSLTCAVSGASFSGHYWTW |
| | | helix3-hinge_10SA7- | IRQPPGKGLEWIGEIDHTGSTNYEPSLRSRVTISVDTS |
| | | VH | KNQFSLNLKSVTAADTAVYYCARGGQGGYDWGHYH |
| | | (hinge-helix3-hinge = | GLDVWGQGTTVTVSSASTKGPSVFPLAPSSKSTSGG |
| | | HHL3) | TAALGCLVKDYFPEPVTVSWNSGALTSGVHTFPA |
| | | | VLQSSGLYSLSSVVTVPSSSLGTQTYICNVNHKPS |
| | | | 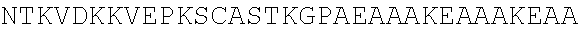 |
| | | | 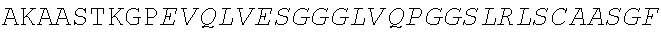 |
| | | | TFSSYAMHWVRQAPGKGLEWVASISSTSGSKYYADSV |
| | | | KGRFTISRDNSKNTLYLQMNSLRAEDTAVYSCAKTYY |
| | | | DFWSGYYTFDYWGQGTLVTVSSASTKGPSVFPLAPS |
| | | | SKSTSGGTAALGCLVKDYFPEPVTVSWNSGALTS |
| | | | GVHTFPAVLQSSGLYSLSSVVTVPSSSLGTQTYICN |
| | | | VNHKPSNTKVDKKVEPKSCDKTHTCPPCPAPEAA |
| | | | GGPSVFLFPPKPKDTLMISRTPEVTCVVVDVSHED |
| | | | PEVKFNWYVDGVEVHNAKTKPREEQYNSTYRVV |
| | | | SVLTVLHQDWLNGKEYKCKVSNKALGAPIEKTIS |
| | | | KAKGQPREPQVYTLPPSREEMTKNQVSLTCLVKG |
| | | | FYPSDIAVEWESNGQPENNYKTTPPVLDSDGSFFL |
| | | | YSKLTVDKSRWQQGNVFSCSVMHEALHNHYTQK |
| | | | SLSLSPGK* |
| 16 | 13032 | 1RC07-VL_hinge- | SYVLTQPPSVSVSPGQTASITCSGDKVGHKYASWYQQ |
| | | helix3-hinge_10SA7- | KPGQSPVLVIYEDSQRPSGIPVRFSGSNSGNTATLTIS |
| | | VL | GTQAMDEADYYCQAWDSSTDVVFGGGTKLTVLGQP |
| | | (hinge-helix3-hinge = | KAAPSVTLFPPSSEELQANKATLVCLISDFYPGAV |
| | | HHL3) | TVAWKADSSPVKAGVETTTPSKQSNNKYAASSYL |
| | | | 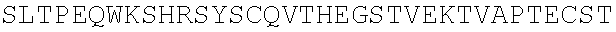 |
| | | | 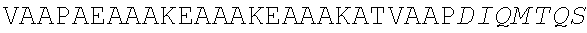 |
| | | | PSSLSASVGDRVTITCRASQSISSYLNWYQQKPGKAPK |
| | | | LLIYAASSLQSGVPSRFSGSGSGTDFTLTISSLQPEDFA |
| | | | TYYCQQSYSTPLTFGGGTKVEIKRTVAAPSVFIFPPS |
| | | | DEQLKSGTASVVCLLNNFYPREAKVQWKVDNAL |
| | | | QSGNSQESVTEQDSKDSTYSLSSTLTLSKADYEKH |
| | | | KVYACEVTHQGLSSPVTKSFNRGEC* |

Amino acid sequence for the constructs used for WNT agonists as DFabOC-Ig

| | | | |
|---|---|---|---|
| 17 | 13101 | YW211.31.57-VH_ | EVQLVESGGGLVQPGGSLRLSCAASGFTFTSYYISWV |
| | | 20mer_18R5-VL | RQAPGKGLEWVAEISPYSGSTYYADSVKGRFTISADTS |
| | | | KNTAYLQMNSLRAEDTAVYYCALRARPPIRLHPRGSV |
| | | | MDYWGQGTLVTVSSASTKGPSVFPLAPSSKSTSGGT |
| | | | AALGCLVKDYFPEPVTVSWNSGALTSGVHTFPAV |
| | | | LQSSGLYSLSSVVTVPSSSLGTQTYICNVNHKPSNT |
| | | | 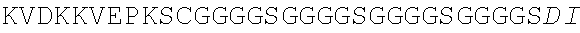 |
| | | | ELTQPPSVSVAPGQTARISCSGDNIGSFYVHWYQQKP |
| | | | GQAPVLVIYDKSNRPSGIPERFSGSNSGNTATLTISGT |
| | | | QAEDEADYYCQSYANTLSLVFGGGTKLTVLGQPKAA |
| | | | PSVTLFPPSSEELQANKATLVCLISDFYPGAVTVA |
| | | | WKADSSPVKAGVETTTPSKQSNNKYAASSYLSLT |
| | | | PEQWKSHRSYSCQVTHEGSTVEKTVAPTECS* |
| 18 | 13102 | YW211.31.57- | EVQLVESGGGLVQPGGSLRLSCAASGFTFTSYYISWV |
| | | VH_FcHinge_18R5- | RQAPGKGLEWVAEISPYSGSTYYADSVKGRFTISADTS |
| | | VL | KNTAYLQMNSLRAEDTAVYYCALRARPPIRLHPRGSV |
| | | | MDYWGQGTLVTVSSASTKGPSVFPLAPSSKSTSGGT |
| | | | AALGCLVKDYFPEPVTVSWNSGALTSGVHTFPAV |
| | | | LQSSGLYSLSSVVTVPSSSLGTQTYICNVNHKPSNT |
| | | | 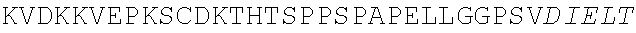 |
| | | | QPPSVSVAPGQTARISCSGDNIGSFYVHWYQQKPGQ |
| | | | APVLVIYDKSNRPSGIPERFSGSNSGNTATLTISGTQAE |
| | | | DEADYYCQSYANTLSLVFGGGTKLTVLGQPKAAPSV |
| | | | TLFPPSSEELQANKATLVCLISDFYPGAVTVAWKA |
| | | | DSSPVKAGVETTTPSKQSNNKYAASSYLSLTPEQ |
| | | | WKSHRSYSCQVTHEGSTVEKTVAPTECS* |
| 19 | 13103 | YW211.31.57- | EVQLVESGGGLVQPGGSLRLSCAASGFTFTSYYISWV |
| | | VH_UpperHinge- | RQAPGKGLEWVAEISPYSGSTYYADSVKGRFTISADTS |
| | | Helix3- | KNTAYLQMNSLRAEDTAVYYCALRARPPIRLHPRGSV |
| | | UpperHinge_18R5- | MDYWGQGTLVTVSSASTKGPSVFPLAPSSKSTSGGT |
| | | VL | AALGCLVKDYFPEPVTVSWNSGALTSGVHTFPAV |
| | | | LQSSGLYSLSSVVTVPSSSLGTQTYICNVNHKPSNT |
| | | | 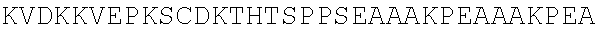 |
| | | | 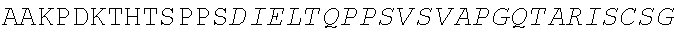 |
| | | | DNIGSFYVHWYQQKPGQAPVLVIYDKSNRPSGIPERF |
| | | | SGSNSGNTATLTISGTQAEDEADYYCQSYANTLSLVFG |
| | | | GGTKLTVLGQPKAAPSVTLFPPSSEELQANKATLV |
| | | | CLISDFYPGAVTVAWKADSSPVKAGVETTTPSKQ |
| | | | SNNKYAASSYLSLTPEQWKSHRSYSCQVTHEGST |
| | | | VEKTVAPTECS* |
| 20 | 13104 | YW211.31.57- | DIQMTQSPSSLSASVGDRVTITCRASQDVSTAVAWYQ |
| | | VL_20mer_18R5-VH | QKPGKAPKLLIYSASFLYSGVPSRFSGSGSGTDFTLTIS |
| | | | SLQPEDFATYYCQQSYTTPPTFGQGTKVEIKRTVAAP |
| | | | SVFIFPPSDEQLKSGTASVVCLLNNFYPREAKVQW |
| | | | KVDNALQSGNSQESVTEQDSKDSTYSLSSTLTLSK |
| | | | 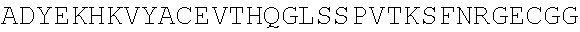 |
| | | | 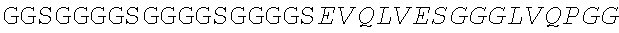 |
| | | | SLRLSCAASGFTFSHYTLSWVRQAPGKGLEWVSVISG |
| | | | DGSYTYYADSVKGRFTISSDNSKNTLYLQMNSLRAED |
| | | | TAVYYCARNFIKYVFANWGQGTLVTVSSASTKGPSV |
| | | | FPLAPSSKSTSGGTAALGCLVKDYFPEPVTVSWNS |
| | | | GALTSGVHTFPAVLQSSGLYSLSSVVTVPSSSLGT |
| | | | QTYICNVNHKPSNTKVDKKVEPKSCDKTHTCPPC |
| | | | PAPEAAGGPSVFLFPPKPKDTLMISRTPEVTCVVV |
| | | | DVSHEDPEVKFNWYVDGVEVHNAKTKPREEQYN |
| | | | STYRVVSVLTVLHQDWLNGKEYKCKVSNKALGA |
| | | | PIEKTISKAKGQPREPQVYTLPPSREEMTKNQVSLT |
| | | | CLVKGFYPSDIAVEWESNGQPENNYKTTPPVLDS |
| | | | DGSFFLYSKLTVDKSRWQQGNVFSCSVMHEALH |
| | | | NHYTQKSLSLSPGK* |
| 21 | 13105 | YW211.31.57- | DIQMTQSPSSLSASVGDRVTITCRASQDVSTAVAWYQ |
| | | VL_kHinge-Helix3- | QKPGKAPKLLIYSASFLYSGVPSRFSGSGSGTDFTLTIS |
| | | UpperHinge_18R5- | SLQPEDFATYYCQQSYTTPPTFGQGTKVEIKRTVAAP |
| | | VH | SVFIFPPSDEQLKSGTASVVCLLNNFYPREAKVQW |
| | | | KVDNALQSGNSQESVTEQDSKDSTYSLSSTLTLSK |
| | | | 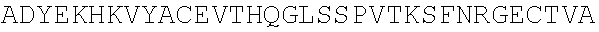 |
| | | | 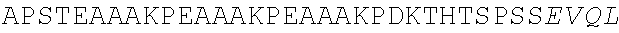 |
| | | | VESGGGLVQPGGSLRLSCAASGFTFSHYTLSWVRQAP |
| | | | GKGLEWVSVISGDGSYTYYADSVKGRFTISSDNSKNTL |
| | | | YLQMNSLRAEDTAVYYCARNFIKYVFANWGQGTLVT |
| | | | VSSASTKGPSVFPLAPSSKSTSGGTAALGCLVKDY |
| | | | FPEPVTVSWNSGALTSGVHTFPAVLQSSGLYSLSS |
| | | | VVTVPSSSLGTQTYICNVNHKPSNTKVDKKVEPKS |
| | | | CDKTHTCPPCPAPEAAGGPSVFLFPPKPKDTLMIS |
| | | | RTPEVTCVVVDVSHEDPEVKFNWYVDGVEVHNA |
| | | | KTKPREEQYNSTYRVVSVLTVLHQDWLNGKEYK |
| | | | CKVSNKALGAPIEKTISKAKGQPREPQVYTLPPSR |
| | | | EEMTKNQVSLTCLVKGFYPSDIAVEWESNGQPEN |
| | | | NYKTTPPVLDSDGSFFLYSKLTVDKSRWQQGNVF |
| | | | SCSVMHEALHNHYTQKSLSLSPGK* |

Amino acid sequence for the constructs used for WNT agonists as DFabIC-Ig

| | | | |
|---|---|---|---|
| 22 | 13106 | YW211.31.57-VL_ | DIQMTQSPSSLSASVGDRVTITCRASQDVSTAVAWYQ |
| | | 20mer_18R5-VH | QKPGKAPKLLIYSASFLYSGVPSRFSGSGSGTDFTLTIS |
| | | | SLQPEDFATYYCQQSYTTPPTFGQGTKVEIKRTVAAP |
| | | | SVFIFPPSDEQLKSGTASVVCLLNNFYPREAKVQW |
| | | | KVDNALQSGNSQESVTEQDSKDSTYSLSSTLTLSK |
| | | | 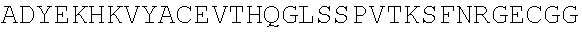 |
| | | | 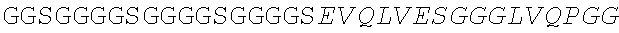 |
| | | | SLRLSCAASGFTFSHYTLSWVRQAPGKGLEWVSVISG |
| | | | DGSYTYYADSVKGRFTISSDNSKNTLYLQMNSLRAED |
| | | | TAVYYCARNFIKYVFANWGQGTLVTVSSASTKGPSV |
| | | | FPLAPSSKSTSGGTAALGCLVKDYFPEPVTVSWNS |
| | | | GALTSGVHTFPAVLQSSGLYSLSSVVTVPSSSLGT |
| | | | QTYICNVNHKPSNTKVDKKVEPKSC* |
| 23 | 13107 | YW211.31.57-VL_ | DIQMTQSPSSLSASVGDRVTITCRASQDVSTAVAWYQ |
| | | kHinge-Helix3- | QKPGKAPKLLIYSASFLYSGVPSRFSGSGSGTDFTLTIS |
| | | UpperHinge_18R5- | SLQPEDFATYYCQQSYTTPPTFGQGTKVEIKRTVAAP |
| | | VH | SVFIFPPSDEQLKSGTASVVCLLNNFYPREAKVQW |
| | | | KVDNALQSGNSQESVTEQDSKDSTYSLSSTLTLSK |
| | | | 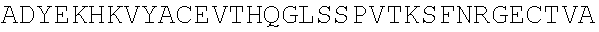 |
| | | | 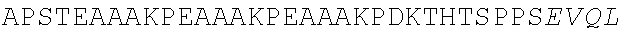 |
| | | | VESGGGLVQPGGSLRLSCAASGFTFSHYTLSWVRQAP |
| | | | GKGLEWVSVISGDGSYTYYADSVKGRFTISSDNSKNTL |
| | | | YLQMNSLRAEDTAVYYCARNFIKYVFANWGQGTLVT |
| | | | VSSASTKGPSVFPLAPSSKSTSGGTAALGCLVKDY |
| | | | FPEPVTVSWNSGALTSGVHTFPAVLQSSGLYSLSS |
| | | | VVTVPSSSLGTQTYICNVNHKPSNTKVDKKVEPKS |
| | | | C* |
| 24 | 13108 | YW211.31.57- | EVQLVESGGGLVQPGGSLRLSCAASGFTFTSYYISWV |
| | | VH_20mer_18R5-VL | RQAPGKGLEWVAEISPYSGSTYYADSVKGRFTISADTS |
| | | | KNTAYLQMNSLRAEDTAVYYCALRARPPIRLHPRGSV |
| | | | MDYWGQGTLVTVSSASTKGPSVFPLAPSSKSTSGG |
| | | | TAALGCLVKDYFPEPVTVSWNSGALTSGVHTFPA |
| | | | VLQSSGLYSLSSVVTVPSSSLGTQTYICNVNHKPS |
| | | | 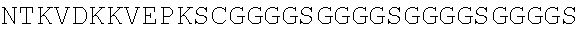 |
| | | | DIELTQPPSVSVAPGQTARISCSGDNIGSFYVHWYQQ |
| | | | KPGQAPVLVIYDKSNRPSGIPERFSGSNSGNTATLTIS |
| | | | GTQAEDEADYYCQSYANTLSLVFGGGTKLTVLGQPK |
| | | | AAPSVTLFPPSSEELQANKATLVCLISDFYPGAVT |
| | | | VAWKADSSPVKAGVETTTPSKQSNNKYAASSYLS |
| | | | LTPEQWKSHRSYSCQVTHEGSTVEKTVAPTECSD |
| | | | KTHTCPPCPAPEAAGGPSVFLFPPKPKDTLMISRTP |
| | | | EVTCVVVDVSHEDPEVKFNWYVDGVEVHNAKTK |
| | | | PREEQYNSTYRVVSVLTVLHQDWLNGKEYKCKV |
| | | | SNKALGAPIEKTISKAKGQPREPQVYTLPPSREEM |
| | | | TKNQVSLTCLVKGFYPSDIAVEWESNGQPENNYK |
| | | | TTPPVLDSDGSFFLYSKLTVDKSRWQQGNVFSCS |
| | | | VMHEALHNHYTQKSLSLSPGK* |
| 25 | 13109 | YW211.31.57-VH_ | EVQLVESGGGLVQPGGSLRLSCAASGFTFTSYYISWV |
| | | FcHinge_18R5-VL | RQAPGKGLEWVAEISPYSGSTYYADSVKGRFTISADTS |
| | | | KNTAYLQMNSLRAEDTAVYYCALRARPPIRLHPRGSV |
| | | | MDYWGQGTLVTVSSASTKGPSVFPLAPSSKSTSGG |
| | | | TAALGCLVKDYFPEPVTVSWNSGALTSGVHTFPA |
| | | | VLQSSGLYSLSSVVTVPSSSLGTQTYICNVNHKPS |
| | | | 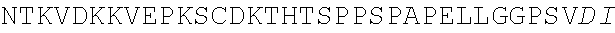 |
| | | | ELTQPPSVSVAPGQTARISCSGDNIGSFYVHWYQQKP |
| | | | GQAPVLVIYDKSNRPSGIPERFSGSNSGNTATLTISGT |
| | | | QAEDEADYYCQSYANTLSLVFGGGTKLTVLGQPKAA |
| | | | PSVTLFPPSSEELQANKATLVCLISDFYPGAVTVA |
| | | | WKADSSPVKAGVETTTPSKQSNNKYAASSYLSLT |
| | | | PEQWKSHRSYSCQVTHEGSTVEKTVAPTECSDKT |
| | | | HTCPPCPAPEAAGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALGAPIEKTISKAKGQPREPQVYTLPPSREEMTK |
| | | | NQVSLTCLVKGFYPSDIAVEWESNGQPENNYKTT |
| | | | PPVLDSDGSFFLYSKLTVDKSRWQQGNVFSCSVM |
| | | | HEALHNHYTQKSLSLSPGK* |
| 26 | 13110 | YW211.31.57- | EVQLVESGGGLVQPGGSLRLSCAASGFTFTSYYISWV |
| | | VL_kHinge-Helix3- | RQAPGKGLEWVAEISPYSGSTYYADSVKGRFTISADTS |
| | | UpperHinge_18R5- | KNTAYLQMNSLRAEDTAVYYCALRARPPIRLHPRGSV |
| | | VH | MDYWGQGTLVTVSSASTKGPSVFPLAPSSKSTSGG |
| | | | TAALGCLVKDYFPEPVTVSWNSGALTSGVHTFPA |
| | | | VLQSSGLYSLSSVVTVPSSSLGTQTYICNVNHKPS |
| | | | 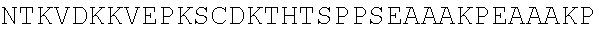 |
| | | | 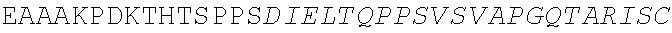 |
| | | | SGDNIGSFYVHWYQQKPGQAPVLVIYDKSNRPSGIPE |
| | | | RFSGSNSGNTATLTISGTQAEDEADYYCQSYANTLSLV |
| | | | FGGGTKLTVLGQPKAAPSVTLFPPSSEELQANKAT |
| | | | LVCLISDFYPGAVTVAWKADSSPVKAGVETTTPS |
| | | | KQSNNKYAASSYLSLTPEQWKSHRSYSCQVTHEG |
| | | | STVEKTVAPTECSDKTHTCPPCPAPEAAGGPSVFL |
| | | | FPPKPKDTLMISRTPEVTCVVVDVSHEDPEVKFN |
| | | | WYVDGVEVHNAKTKPREEQYNSTYRVVSVLTVL |
| | | | HQDWLNGKEYKCKVSNKALGAPIEKTISKAKGQP |
| | | | REPQVYTLPPSREEMTKNQVSLTCLVKGFYPSDIA |
| | | | VEWESNGQPENNYKTTPPVLDSDGSFFLYSKLTV |
| | | | DKSRWQQGNVFSCSVMHEALHNHYTQKSLSLSP |
| | | | GK* |

Amino acid sequence for the constructs used for WNT-agonists as DFabIC-Ig

| | | | |
|---|---|---|---|
| 27 | 13111 | pembro- | QVQLVQSGVEVKKPGASVKVSCKASGYTFTNYYMYW |
| | | VH_10mer_18R5-VL | VRQAPGQGLEWMGGINPSNGGTNFNEKFKNRVTLT |
| | | | TDSSTTTAYMELKSLQFDDTAVYYCARRDYRFDMGF |
| | | | DYWGQGTTVTVSSASTKGPSVFPLAPSSKSTSGGTA |
| | | | ALGCLVKDYFPEPVTVSWNSGALTSGVHTFPAVL |
| | | | QSSGLYSLSSVVTVPSSSLGTQTYICNVNHKPSNT |
| | | | 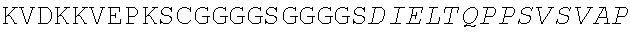 |
| | | | GQTARISCSGDNIGSFYVHWYQQKPGQAPVLVIYDKS |
| | | | NRPSGIPERFSGSNSGNTATLTISGTQAEDEADYYCQS |
| | | | YANTLSLVFGGGTKLTVLGQPKAAPSVTLFPPSSEEL |
| | | | QANKATLVCLISDFYPGAVTVAWKADSSPVKAG |
| | | | VETTTPSKQSNNKYAASSYLSLTPEQWKSHRSYSC |
| | | | QVTHEGSTVEKTVAPTECS* |
| 28 | 13112 | pembro- | EIVLTQSPATLSLSPGERATLSCRASKGVSTSGYSYLH |
| | | VL_10mer_18R5VH | WYQQKPGQAPRLLIYLASYLESGVPARFSGSGSGTDF |
| | | | TLTISSLEPEDFAVYYCQHSRDLPLTFGGGTKVEIKRT |
| | | | VAAPSVFIFPPSDEQLKSGTASVVCLLNNFYPREA |
| | | | KVQWKVDNALQSGNSQESVTEQDSKDSTYSLSST |
| | | | LTLSKADYEKHKVYACEVTHQGLSSPVTKSFNRG |
| | | | 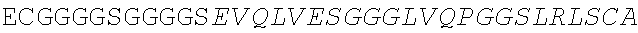 |
| | | | ASGFTFSHYTLSWVRQAPGKGLEWVSVISGDGSYTYY |
| | | | ADSVKGRFTISSDNSKNTLYLQMNSLRAEDTAVYYCA |
| | | | RNFIKYVFANWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCDKTHTCPPCPAPEAAGG |
| | | | PSVFLFPPKPKDTLMISRTPEVTCVVVDVSHEDPE |
| | | | VKFNWYVDGVEVHNAKTKPREEQYNSTYRVVSV |
| | | | LTVLHQDWLNGKEYKCKVSNKALGAPIEKTISKA |
| | | | KGQPREPQVYTLPPSREEMTKNQVSLTCLVKGFY |
| | | | PSDIAVEWESNGQPENNYKTTPPVLDSDGSFFLYS |
| | | | KLTVDKSRWQQGNVFSCSVMHEALHNHYTQKSL |
| | | | SLSPGK* |
| 29 | 13113 | pembro- | QVQLVQSGVEVKKPGASVKVSCKASGYTFTNYYMYW |
| | | 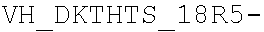 | VRQAPGQGLEWMGGINPSNGGTNFNEKFKNRVTLT |
| | | VL | TDSSTTTAYMELKSLQFDDTAVYYCARRDYRFDMGF |
| | | | DYWGQGTTVTVSSASTKGPSVFPLAPSSKSTSGGTA |
| | | | ALGCLVKDYFPEPVTVSWNSGALTSGVHTFPAVL |
| | | | QSSGLYSLSSVVTVPSSSLGTQTYICNVNHKPSNT |
| | | | 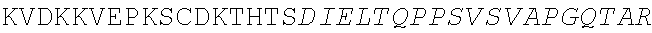 |
| | | | ISCSGDNIGSFYVHWYQQKPGQAPVLVIYDKSNRPSG |
| | | | IPERFSGSNSGNTATLTISGTQAEDEADYYCQSYANTL |
| | | | SLVFGGGTKLTVLGQPKAAPSVTLFPPSSEELQANK |
| | | | ATLVCLISDFYPGAVTVAWKADSSPVKAGVETTT |
| | | | PSKQSNNKYAASSYLSLTPEQWKSHRSYSCQVTH |
| | | | EGSTVEKTVAPTECS* |
| 30 | 13114 | pembro- | EIVLTQSPATLSLSPGERATLSCRASKGVSTSGYSYLH |
| | | 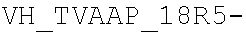 | WYQQKPGQAPRLLIYLASYLESGVPARFSGSGSGTDF |
| | | VH | TLTISSLEPEDFAVYYCQHSRDLPLTFGGGTKVEIKRT |
| | | | VAAPSVFIFPPSDEQLKSGTASVVCLLNNFYPREA |
| | | | KVQWKVDNALQSGNSQESVTEQDSKDSTYSLSST |
| | | | LTLSKADYEKHKVYACEVTHQGLSSPVTKSFNRG |
| | | | 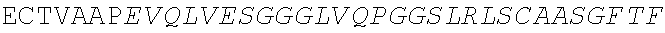 |
| | | | SHYTLSWVRQAPGKGLEWVSVISGDGSYTYYADSVKG |
| | | | RFTISSDNSKNTLYLQMNSLRAEDTAVYYCARNFIKYV |
| | | | FANWGQGTLVTVSSASTKGPSVFPLAPSSKSTSGGT |
| | | | AALGCLVKDYFPEPVTVSWNSGALTSGVHTFPAV |
| | | | LQSSGLYSLSSVVTVPSSSLGTQTYICNVNHKPSNT |
| | | | KVDKKVEPKSCDKTHTCPPCPAPEAAGGPSVFLFP |
| | | | PKPKDTLMISRTPEVTCVVVDVSHEDPEVKFNWY |
| | | | VDGVEVHNAKTKPREEQYNSTYRVVSVLTVLHQ |
| | | | DWLNGKEYKCKVSNKALGAPIEKTISKAKGQPRE |
| | | | PQVYTLPPSREEMTKNQVSLTCLVKGFYPSDIAVE |
| | | | WESNGQPENNYKTTPPVLDSDGSFFLYSKLTVDK |
| | | | SRWQQGNVFSCSVMHEALHNHYTQKSLSLSPGK* |
| 31 | 13115 | pembro- | QVQLVQSGVEVKKPGASVKVSCKASGYTFTNYYMYW |
| | | 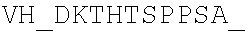 | VRQAPGQGLEWMGGINPSNGGTNFNEKFKNRVTLT |
| | | 18R5-VL | TDSSTTTAYMELKSLQFDDTAVYYCARRDYRFDMGF |
| | | | DYWGQGTTVTVSSASTKGPSVFPLAPSSKSTSGGTA |
| | | | ALGCLVKDYFPEPVTVSWNSGALTSGVHTFPAVL |
| | | | QSSGLYSLSSVVTVPSSSLGTQTYICNVNHKPSNT |
| | | | 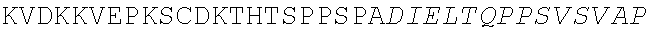 |
| | | | GQTARISCSGDNIGSFYVHWYQQKPGQAPVLVIYDKS |
| | | | NRPSGIPERFSGSNSGNTATLTISGTQAEDEADYYCQS |
| | | | YANTLSLVFGGGTKLTVLGQPKAAPSVTLFPPSSEEL |
| | | | QANKATLVCLISDFYPGAVTVAWKADSSPVKAG |
| | | | VETTTPSKQSNNKYAASSYLSLTPEQWKSHRSYSC |
| | | | QVTHEGSTVEKTVAPTECS |
| 32 | 13116 | pembro- | EIVLTQSPATLSLSPGERATLSCRASKGVSTSGYSYLH |
| | | 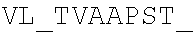 | WYQQKPGQAPRLLIYLASYLESGVPARFSGSGSGTDF |
| | | 18R5-VH | TLTISSLEPEDFAVYYCQHSRDLPLTFGGGTKVEIKRT |
| | | | VAAPSVFIFPPSDEQLKSGTASVVCLLNNFYPREA |
| | | | KVQWKVDNALQSGNSQESVTEQDSKDSTYSLSST |
| | | | LTLSKADYEKHKVYACEVTHQGLSSPVTKSFNRG |
| | | | 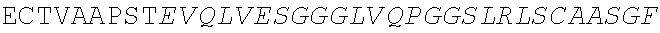 |
| | | | TFSHYTLSWVRQAPGKGLEWVSVISGDGSYTYYADSV |
| | | | KGRFTISSDNSKNTLYLQMNSLRAEDTAVYYCARNFIK |
| | | | YVFANWGQGTLVTVSSASTKGPSVFPLAPSSKSTSG |
| | | | GTAALGCLVKDYFPEPVTVSWNSGALTSGVHTFP |
| | | | AVLQSSGLYSLSSVVTVPSSSLGTQTYICNVNHKP |
| | | | SNTKVDKKVEPKSCDKTHTCPPCPAPEAAGGPSV |
| | | | FLFPPKPKDTLMISRTPEVTCVVVDVSHEDPEVKF |
| | | | NWYVDGVEVHNAKTKPREEQYNSTYRVVSVLTV |
| | | | LHQDWLNGKEYKCKVSNKALGAPIEKTISKAKGQ |
| | | | PREPQVYTLPPSREEMTKNQVSLTCLVKGFYPSDI |
| | | | AVEWESNGQPENNYKTTPPVLDSDGSFFLYSKLT |
| | | | VDKSRWQQGNVFSCSVMHEALHNHYTQKSLSLS |
| | | | PGK* |
| 33 | 13119 | 18R5- | EVQLVESGGGLVQPGGSLRLSCAASGFTFSHYTLSWV |
| | | 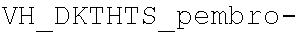 | RQAPGKGLEWVSVISGDGSYTYYADSVKGRFTISSDN |
| | | VL | SKNTLYLQMNSLRAEDTAVYYCARNFIKYVFANWGQ |
| | | | GTLVTVSSASTKGPSVFPLAPSSKSTSGGTAALGCL |
| | | | VKDYFPEPVTVSWNSGALTSGVHTFPAVLQSSGL |
| | | | YSLSSVVTVPSSSLGTQTYICNVNHKPSNTKVDKK |
| | | | 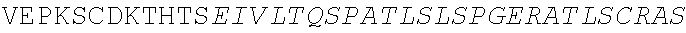 |
| | | | KGVSTSGYSYLHWYQQKPGQAPRLLIYLASYLESGVP |
| | | | ARFSGSGSGTDFTLTISSLEPEDFAVYYCQHSRDLPLT |
| | | | FGGGTKVEIKRTVAAPSVFIFPPSDEQLKSGTASVV |
| | | | CLLNNFYPREAKVQWKVDNALQSGNSQESVTEQ |
| | | | DSKDSTYSLSSTLTLSKADYEKHKVYACEVTHQG |
| | | | LSSPVTKSFNRGEC* |
| 34 | 13120 | 18R5- | DIELTQPPSVSVAPGQTARISCSGDNIGSFYVHWYQQ |
| | | 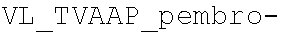 | KPGQAPVLVIYDKSNRPSGIPERFSGSNSGNTATLTIS |
| | | VH | GTQAEDEADYYCQSYANTLSLVFGGGTKLTVLGQPK |
| | | | AAPSVTLFPPSSEELQANKATLVCLISDFYPGAVT |
| | | | VAWKADSSPVKAGVETTTPSKQSNNKYAASSYLS |
| | | | 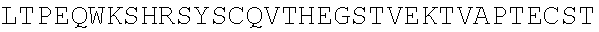 |
| | | | 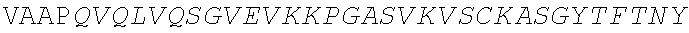 |
| | | | YMYWVRQAPGQGLEWMGGINPSNGGTNFNEKFKN |
| | | | RVTLTTDSSTTTAYMELKSLQFDDTAVYYCARRDYRF |
| | | | DMGFDYWGQGTTVTVSSASTKGPSVFPLAPSSKSTS |
| | | | GGTAALGCLVKDYFPEPVTVSWNSGALTSGVHTF |
| | | | PAVLQSSGLYSLSSVVTVPSSSLGTQTYICNVNHK |
| | | | PSNTKVDKKVEPKSCDKTHTCPPCPAPEAAGGPS |
| | | | VFLFPPKPKDTLMISRTPEVTCVVVDVSHEDPEVK |
| | | | FNWYVDGVEVHNAKTKPREEQYNSTYRVVSVLT |
| | | | VLHQDWLNGKEYKCKVSNKALGAPIEKTISKAKG |
| | | | QPREPQVYTLPPSREEMTKNQVSLTCLVKGFYPSD |
| | | | IAVEWESNGQPENNYKTTPPVLDSDGSFFLYSKLT |
| | | | VDKSRWQQGNVFSCSVMHEALHNHYTQKSLSLS |
| | | | PGK |
| 35 | 13121 | 18R5- | EVQLVESGGGLVQPGGSLRLSCAASGFTFSHYTLSWV |
| | | 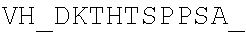 | RQAPGKGLEWVSVISGDGSYTYYADSVKGRFTISSDN |
| | | pembro-VL | SKNTLYLQMNSLRAEDTAVYYCARNFIKYVFANWGQ |
| | | | GTLVTVSSASTKGPSVFPLAPSSKSTSGGTAALGCL |
| | | | VKDYFPEPVTVSWNSGALTSGVHTFPAVLQSSGL |
| | | | YSLSSVVTVPSSSLGTQTYICNVNHKPSNTKVDKK |
| | | | 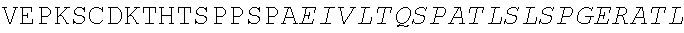 |
| | | | SCRASKGVSTSGYSYLHWYQQKPGQAPRLLIYLASYLE |
| | | | SGVPARFSGSGSGTDFTLTISSLEPEDFAVYYCQHSRD |
| | | | LPLTFGGGTKVEIKRTVAAPSVFIFPPSDEQLKSGTA |
| | | | SVVCLLNNFYPREAKVQWKVDNALQSGNSQESV |
| | | | TEQDSKDSTYSLSSTLTLSKADYEKHKVYACEVT |
| | | | HQGLSSPVTKSFNRGEC* |
| 36 | 13122 | 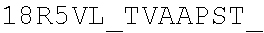 | DIELTQPPSVSVAPGQTARISCSGDNIGSFYVHWYQQ |
| | | pembro-VH | KPGQAPVLVIYDKSNRPSGIPERFSGSNSGNTATLTIS |
| | | | GTQAEDEADYYCQSYANTLSLVFGGGTKLTVLGQPK |
| | | | AAPSVTLFPPSSEELQANKATLVCLISDFYPGAVT |
| | | | VAWKADSSPVKAGVETTTPSKQSNNKYAASSYLS |
| | | | 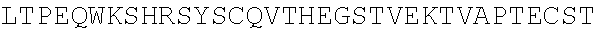 |
| | | | 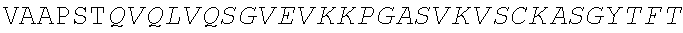 |
| | | | NYYMYWVRQAPGQGLEWMGGINPSNGGTNFNEKF |
| | | | KNRVTLTTDSSTTTAYMELKSLQFDDTAVYYCARRDY |
| | | | RFDMGFDYWGQGTTVTVSSASTKGPSVFPLAPSSKS |
| | | | TSGGTAALGCLVKDYFPEPVTVSWNSGALTSGVH |
| | | | TFPAVLQSSGLYSLSSVVTVPSSSLGTQTYICNVNH |
| | | | KPSNTKVDKKVEPKSCDKTHTCPPCPAPEAAGGP |
| | | | SVFLFPPKPKDTLMISRTPEVTCVVVDVSHEDPEV |
| | | | KFNWYVDGVEVHNAKTKPREEQYNSTYRVVSVL |
| | | | TVLHQDWLNGKEYKCKVSNKALGAPIEKTISKAK |
| | | | GQPREPQVYTLPPSREEMTKNQVSLTCLVKGFYPS |
| | | | DIAVEWE SNGQPENNYKTTPPVLDSDGSFFLYSKL |
| | | | TVDKSRWQQGNVFSCSVMHEALHNHYTQKSLSL |
| | | | SPGK |

Amino acid sequence for the constructs used for CrossFIT-Ig

| | | | |
|---|---|---|---|
| 37 | 09273 | 1RC07-VL | SYVLTQPPSVSVSPGQTASITCSGDKVGHKYASWYQQ |
| | | | KPGQSPVLVIYEDSQRPSGIPVRFSGSNSGNTATLTIS |
| | | | GTQAMDEADYYCQAWDSSTDVVFGGGTKLTVLGQP |
| | | | KAAPSVTLFPPSSEELQANKATLVCLISDFYPGAV |
| | | | TVAWKADSSPVKAGVETTTPSKQSNNKYAASSYL |
| | | | SLTPEQWKSHRSYSCQVTHEGSTVEKTVAPTECS* |
| 38 | 06821 | 10SA7-VH | EVQLVESGGGLVQPGGSLRLSCAASGFTFSSYAMHW |
| | | | VRQAPGKGLEWVASISSTSGSKYYADSVKGRFTISRDN |
| | | | SKNTLYLQMNSLRAEDTAVYSCAKTYYDFWSGYYTFD |
| | | | YWGQGTLVTVSSASTKGPSVFPLAPSSKSTSGGTAA |
| | | | LGCLVKDYFPEPVTVSWNSGALTSGVHTFPAVLQ |
| | | | SSGLYSLSSVVTVPSSSLGTQTYICNVNHKPSNTK |
| | | | VDKKVEPKSCGSGSGHHHHHH* |
| 39 | 14036 | 1RCO7-VH_ | QVQLQQWGAGLLKPSEILSLTCAVSGASFSGHYWTW |
| | | 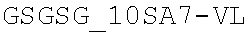 | IRQPPGKGLEWIGEIDIITGSTNYEPSLRSRVTISVDTS |
| | | | KNQFSLNLKSVTAADTAVYYCARGGQGGYDWGHYH |
| | | | GLDVWGQGTTVTVSSASTKGPSVFPLAPSSKSTSGG |
| | | | TAALGCLVKDYFPEPVTVSWNSGALTSGVHTFPA |
| | | | VLQSSGLYSLSSVVTVPSSSLGTQTYICNVNHKPS |
| | | | 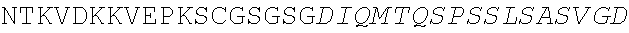 |
| | | | RVTITCRASQSISSYLNWYQQKPGKAPKLLIYAASSLQS |
| | | | GVPSRFSGSGSGTDFTLTISSLQPEDFATYYCQQSYST |
| | | | PLTFGGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVTH |
| | | | QGLSSPVTKSFNRGECDKTHTCPPCPAPEAAGGPS |
| | | | VFLFPPKPKDTLMISRTPEVTCVVVDVSHEDPEVK |
| | | | FNWYVDGVEVHNAKTKPREEQYNSTYRVVSVLT |
| | | | VLHQDWLNGKEYKCKVSNKALGAPIEKTISKAKG |
| | | | QPREPQVYTLPPSREEMTKNQVSLTCLVKGFYPSD |
| | | | IAVEWESNGQPENNYKTTPPVLDSDGSFFLYSKLT |
| | | | VDKSRWQQGNVFSCSVMHEALHNHYTQKSLSLS |
| | | | PGK* |
| 40 | | secretion signal- | MDMRVPAQLLGLLLLWLRGARC |
| | | peptide from Kappa- | |
| | | light-chain | |

Protein expression and purification: Plasmids expressing light-chain and heavy-chain of bispecific tetravalent antibody molecules were co-transfected, using FectoPRO (Polyplus NY USA) for co-expression in Expi293F cells, typically at 80 mL scale, following the standard protocols from the manufacturer. After 4 days of continuous cell growth, media were harvested by centrifugation, and bound to 300 uL of Protein-A resin, washed with PBS (137 mM NaCl, 10 mM phosphate, 2.7 mM KCl; pH 7.4), and eluted with 0.1 M glycine pH 3.3 in the presence of 10% v/v 1M TrisHCl and 1.5M NaCl pH 8.5) under gravity. Elution fractions containing top-three A280 values were pooled and injected into a 24 mL Superdex200-Increase column equilibrated in 2×HBS buffer (40 mM HEPES pH 7.4, 300 mM NaCl). Eluent fractions were examined by SDS-polyacrylamide electrophoresis and those containing molecule of interest were used in cell-based activity assay.

SuperTop Flash (STF) assay: WNT signaling activity was measured using HEK293 cells containing a luciferase gene controlled by a WNT-responsive promoter (Super Top Flash reporter assay, STF) as previously reported (Janda et al., 2017). In brief, cells were seeded at a density of 10,000 per well in 96-well plates 24 hours prior to treatment in the presence of 3 μM IWP2 to inhibit the production of endogenous WNT ligands. The recombinant proteins were then added to the cells in the presence of 20 nM Fc-RSPO2 overnight. A tetravalent bispecific WNT-agonist R2M3-26 was used as positive control. Cells were lysed with Luciferase Cell Culture Lysis Reagent (Promega) and luciferase activity was measured with Luciferase Assay System (Promega) using vendor suggested procedures. For WNT-antagonist assays, no IWP2 was used and the WNT-source was R2M3-26. The inhibitory effect of protein molecules at 5 nM and 10 nM concentrations were measured. Data were plotted as average−/+standard deviation of triplicates Prism (GraphPad Software) or Excel (Microsoft).

### Example 2

### Dual-Fab-Ig (No Cross-Over) Wnt-Agonists

For the design of DFab-Ig Wnt-agonists, an anti-Fzd antibody 1RC07 that recognize CRD of Fzd1, Fzd2, and Fzd7 receptors (WO/2019/126399) and an anti-Lrp6 antibody 10SA7 (WO2019126401), were linked on both light- and heavy-chains without any cross-over (FIG. 1A) using different linkers. DFab-Ig were designed such that various constructs had either 10SA7 as “outer-Fab” and 1RC07 “inner-Fab” and vice versa. Clone ID number and amino acid sequences of designed constructs, along with linker sequences highlighted in gray, are listed in Table 1.

Results of STF assays from eight different tetravalent-bispecific DFab-Ig molecules are shown in FIG. 2. Analyses of STF activity levels reveals the following: (i) compared to the cells-only controls, where no WNT-agonists molecules were added during the assay, all DFab-Ig molecules in which anti-Lrp6 antibody 10SA7 was situated as the “outer-Fab” showed induction of WNT-signaling; (ii) linkers containing helical repeats (see, Table 1, e.g., “HHL”) showed higher WNT-agonism than the one containing flexible linkers (see Table 1, e.g. “HFL”). The activity levels of 10SA7-HHL1-1RC07 and 10SA7-HHL2-1RC07 were two times higher than that of 10SA7-HFL2-1RC07; (iii) increasing levels of WNT-activity was observed with increasing length of the linkers with HHL3 containing DFab-Ig molecules showing highest activity. Given the sizes of Fzd-CRD and Lrp6 extracellular domains, steric considerations could potentially explain the preference in orientation.

### Example 3

### DFabOC-Ig (DFab-Outer Crossover-Ig) Wnt-Agonists

Crossing of light- and heavy-chains has been used in the design of tetravalent bispecific FIT-Ig (Fabs-In-Tandem-Ig; Gong et al (2017) MAbs 9(7): 1118-1128) molecules, whose production requires cotransfection of three plasmid DNAs: one heavy, one light- and one heavy-Fc chain plasmids. A fusion of VL-CL domain of the “outer-Fab” to N-terminus of VH-CH1 domain of the “inner-Fab” (through various linkers) was explored, which in turn is fused to the N-terminus of the Fc domain (DFabOC-Ig; (FIG. 1B). An anti-Fzd antibody 18R5 (Gurney et al., (2012) Proc Natl Acad Sci USA 109(29):11717-22) and an anti-Lrp6 antibody YW211.31.57 were used to illustrate WNT-agonism in DFabOC-Ig format molecules. Clone ID number and amino acid sequences of designed constructs, including linker sequences used highlighted in grey are listed in Table 1.

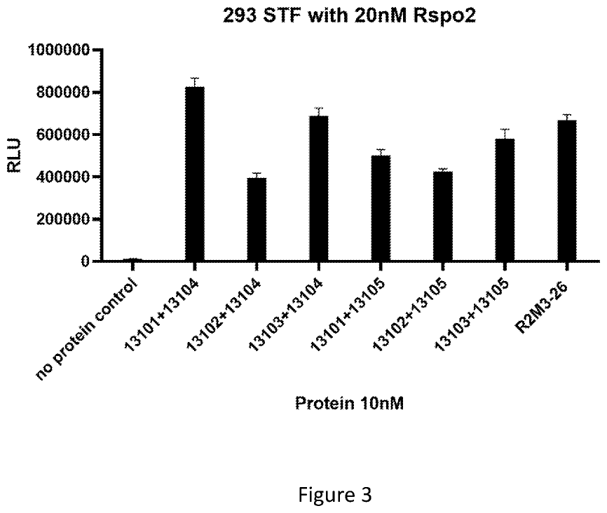

Based on these three light- and two heavy-chains, six DFabIC-Ig tetravalent bispecific molecules were expressed, purified, and fractions containing proteins of expected molecular weights were analyzed for WNT-signaling by STF assay. All six DFabIC-Ig molecules showed significantly elevated levels of WNT-activity compared to the “cells-only” control wells to which no proteins were added (FIG. 3). WNT-agonist activity for the DFabOC-Ig molecules were also either comparable and slightly better (except in one case, where kHinge-Helix3-UpperHinge and UpperHinge-Helix3-UpperHinge linkers were present on light- and heavy-chains, respectively) than that observed for the internal positive control R2M3-26.

### Example 4

### DFabIC-Ig (DFab-Inner Crossover-Ig) WNT-Agonists

As an extension of above described DFabOC-Ig format, tetravalent bispecific antibody molecules in the DFabIC-Ig format were designed, in which the light- and heavy-chains of the “inner Fab” crossed with respect to the Fc domain of the anti-body (FIG. 1C). Heavy-chains DFabIC-Ig have configuration where the heavy-chain of the “outer Fab” is connected to the light-chain of the “inner Fab” which in turn is connected to the N-terminus of the Fc-domain. Similarly, light-chains of DFabIC-Ig have configuration where the light-chain of “outer Fab” is connected to the N-terminus of the heavy-chain of the “inner Fab”. Clone ID number and amino acid sequences of designed constructs, along with linker sequences highlighted in grey are listed in Table 1.

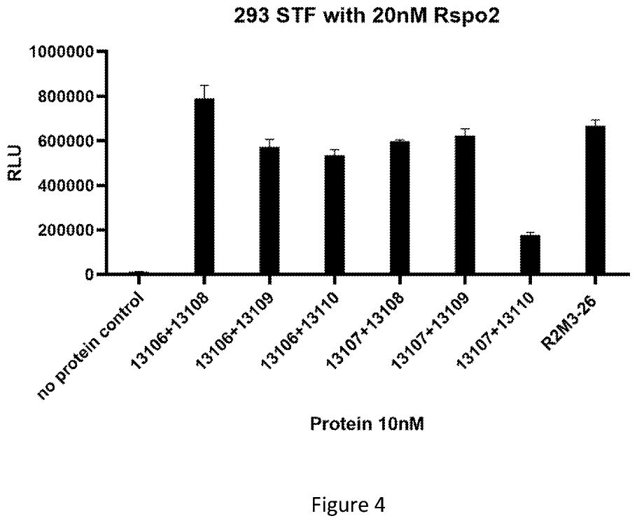

Based on these two light- and three heavy-chains, six DFabIC-Ig tetravalent bispecific molecules were expressed, purified, and fractions containing proteins of expected molecular weights were analyzed for WNT-signaling by STF assay. All six DFabIC-Ig molecules showed significantly elevated levels of WNT-activity compared to the “cells-only” control wells to which no proteins were added (FIG. 4). WNT-agonist activity for the DFabOC-Ig molecules were also either comparable and slightly better (except in one case, where kHinge-Helix3-UpperHinge and UpperHinge-Helix3-UpperHinge linkers were present on light- and heavy-chains, respectively; protein 13107+13110 in FIG. 4) than that observed for the internal positive control R2M3-26.

### Example 5

### DFabOC-Ig (DFab-Outer Crossover-Ig) WNT-Antagonists

In addition to showing that the molecules assembled in DFab-Ig, DFabOC-Ig, and DFabIC-Ig formats can function as WNT-agonists, the ability of tetravalent bispecific molecules designed based on the DFab-Ig platform to functions as WNT-antagonists was also tested. To study WNT-antagonism, we linked the Fab domains of anti-PD-1 antibody pembrolizumab (pembro; marketed as Keytruda® for the treatment of metastatic malignant melanoma; https://www.drugbank.ca/drugs/DB09037) and 18R5 described above DFabOC-Ig format (FIG. 1B) with pembro as “outer Fab” and 18R5 as the “inner Fab” and vice versa. Clone ID number and amino acid sequences of designed constructs, along with linker sequences highlighted in grey are listed in Table 1.

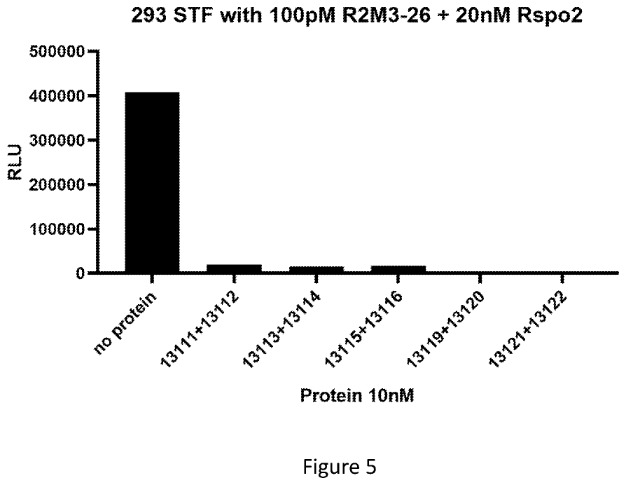

From these twelve constructs listed in Table 1, six light- and heavy-chain pairs were co-transfected, and five proteins were purified. Fractions containing protein of expected molecular weight were tested at 10 nM concentrations for their ability to inhibit WNT-signaling induced by the 100 pM of the surrogate WNT molecule R2M3-26 in the presence of 20 nM RSPO2 (FIG. 5). Proteins assembled in configurations with the Fab domains of pembro and 18R5 as either “outer Fab” or “inner Fab”, both significantly inhibited the WNT-signaling mediated by the R2M3-26 surrogate WNT agonist. Thus, we showed that robust WNT-antagonism could be achieved by tetravalent bispecific antibodies designed based on the DFab-Ig platform.

### Example 6

### WNT-Agonists in CrossFIT-Ig Format

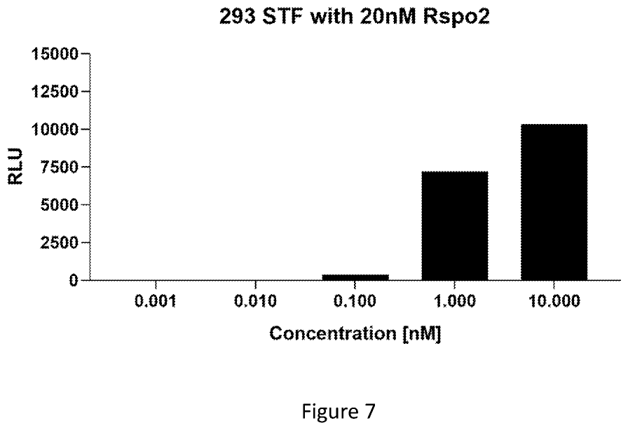

An additional conformation was explored with crisscrossing the “inner Fab” with respect to Fc domain resulting in a new format termed CrossFIT-Ig to generate tetravalent bispecific antibodies (FIG. 6). We used the Fab domains of anti-Fzd antibody 1R07 and anti-Lrp6 antibody 10SA7 to test our hypothesis and co-transfected three plasmids expressing constructs listed in Table 1 for protein expression, purification, and analyzed the fractions containing protein of expected molecular weight using STF assay. Analyses of the results show that molecules designed using CrossFIT-Ig format, in which 1RC07 and 10SA7 are configured as the “outer Fab” and “inner Fab”, respectively, showed WNT-signal activation (FIG. 7) illustrating feasibility of CrossFIT-Ig platform for the design of tetravalent bispecific antibodies.

What is claimed is:

1. A molecule comprising at least two Fab binding domains and an Ig domain, wherein at least one Fab binding domain binds to at least one receptor of a Wnt co-receptor complex.

2. The molecule of claim 1, comprising a first Fab binding domain that binds to a first receptor of the Wnt co-receptor complex and a second Fab binding domain that binds to a second receptor of the Wnt co-receptor complex.

3. The molecule of claim 2 wherein the first Fab binding domain binds to at least one Fzd receptor and the second Fab binding domain binds to at least one LRP receptor.

4. The molecule of claim 3 wherein the first Fab binding domain binds to at least one Fzd receptors selected from the group consisting of Fzd1, Fzd2, Fzd3, Fzd4, Fzd5, Fzd6, Fzd7, Fzd8, Fzd9, and Fzd 10.

5. The molecule of claim 3, wherein the second Fab binding domain binds at least one LRP receptor selected from the group consisting from LRP5, LRP6, and LRP5/6.

6. The molecule of claim 3, wherein the molecule is an agonist of Wnt signaling.

7. The molecule of claim 1, comprising a first Fab binding domain that binds to a receptor of a Wnt co-receptor complex and a second Fab binding domain that binds to a non-Wnt receptor.

8. The molecule of claim 7, wherein the molecule is an antagonist of Wnt signaling.

9. The molecule of claim 1 having a structure selected from the group consisting of the structures presented in FIG. 1A, 1B, 1C and FIG. 6.

10. The molecule of claim 9, wherein the first Fab binding domain (Inner Fab) is fused directly to the second Fab binding domain (Outer Fab).

11. The molecule of claim 9, wherein the Inner Fab is attached to the Outer Fab binding domain with a peptide linker.

12. The molecule of claim 11, wherein the peptide linker is selected from the group consisting of:
a) a hinge-10 mer-hinge (HFL2);
b) a hinge-helix-hinge (HHL);
c) a 20 mer;
d) an FcHinge
e) an upper hinge-helix-upper hinge; and
f) a kappa hinge (khinge).

13. The molecule of claim 12 wherein the peptide linker comprises a sequence set forth in Table 1.

14. The molecule of claim 11, wherein the peptide linker is about 1 amino acid in length to about 30 amino acids in length.

15. The molecule of claim 11, wherein the peptide linker is about 5 amino acids in length to about 15 amino acids in length.

16. A polypeptide comprising or consisting of a polypeptide sequence having at least 70%, at least 80%, at least 90%, at least 95%, at least 98%, or at least 99% sequence identity to a sequence selected from SEQ ID NOs: 1-40.

17. The polypeptide of claim 16, wherein the polypeptide comprises the CDR sequences set forth in any of SEQ ID NOs: 1-40.

18. A molecule comprising two or more polypeptides of claim 16 or claim 17.

19. The molecule of claim 18, wherein the molecule comprises four polypeptides of claim 16 or claim 17.

20. The molecule of claim 19, wherein two of the four polypeptides comprise an Ig or Fc region and the other two of the four polypeptides do not comprises an Ig or Fc region.

21. The molecule of claim 20, wherein each of the four polypeptides comprise two Fab binding domains.

22. The molecule of claim 21, wherein each of the four polypeptides comprises a first Fab binding domain that binds to a first receptor of the Wnt co-receptor complex and a second Fab binding domain that binds to a second receptor of the Wnt co-receptor complex.

23. The molecule of claim 22, wherein the first Fab binding domain binds to at least one Fzd receptor and the second Fab binding domain binds to at least one LRP receptor.

24. A polynucleotide encoding the polypeptide of claim 16 or claim 17.

25. A cell comprising the polynucleotide of claim 24.

26. A method of agonizing the Wnt pathway, comprising contacting a cell with the molecule set forth in any of claim 1-6, 9-15 or 18-23.

27. A method of inhibiting the Wnt pathway, comprising contacting a cell with the molecule set forth in claim 7 or claim 8.

28. A pharmaceutical composition comprising the molecule set forth in any of claim 1-15 or 18-23.

29. A method of treating a disease or disorder where tissue regeneration is desired, comprising providing to a subject in need thereof the molecule set forth in any of claim 1-6, 9-15 or 18-23.
